(19) United States
(12) Patent Application Publication
(10) Pub. No.: US 2010/0184672 A1
(43) Pub. Date: Jul. 22, 2010

(54) PROTEIN C FOR USE IN MAINTAINING HEMOSTASIS

(76) Inventors: Owen J.T. McCarty, Portland, OR (US); Andras Gruber, Portland, OR (US); John h. Griffin, Del Mar, CA (US)

Correspondence Address: KLARQUIST SPARKMAN, LLP, 121 SW SALMON STREET, SUITE 1600, PORTLAND, OR, 97204, US

(21) Appl. No.: 12/665,540

(22) PCT Filed: Jun. 18, 2008
(86) PCT No.: PCT/US08/67321

§ 371 Date: Dec. 18, 2009

Related U.S. Application Data
(60) Provisional application No. 60/944,693, filed on Jun. 18, 2007.

(51) Int. Cl. A61K 38/17; A61K 48/00; A61P 7/04

(52) U.S. Cl. 514/12; 514/44 R

(57) ABSTRACT

It is disclosed herein that protein C functions as a hemostatic agent. Thus, provided is a method of preventing, treating or ameliorating abnormal bleeding in a subject, comprising administering to the subject a protein C polypeptide or polynucleotide. Abnormal bleeding can result from a bleeding disorder, such as hemophilia or a platelet disorder, or from a bleeding episode, such as from a traumatic injury.

### CROSS REFERENCE TO RELATED APPLICATION

This application claims the benefit of priority of U.S. Provisional Application No. 60/944,693, filed Jun. 18, 2007, which is herein incorporated by reference in its entirety.

### STATEMENT OF GOVERNMENT SUPPORT

This invention was made with United States government support under grant HL052246, from the National Heart, Lung, and Blood Institute, National Institutes of Health. The United States government has certain rights in the invention.

### FIELD

This disclosure concerns protein C polypeptides and polynucleotides and their use as hemostatic agents.

### BACKGROUND

Upon damage to vessel walls, exposed extracellular matrix (ECM) proteins and released tissue factor (TF) triggers a series of events that lead to the formation of a hemostatic plug. The essential role of platelet and platelet receptors in this process is illustrated by the profound bleeding exhibited by patients deficient in the major glycoproteins, including GPIb (Bernard-Soulier syndrome) and the integrin α_{IIb}β₃ (Glanzmann thrombasthenia) (A. T. Nurden, J. Thromb. Haemost. 3:1773-1782, 2005; Rao et al. Semin. Thromb. Hemost. 30:525-535, 2004). In addition, the critical role of coagulation factors is evidenced by the severe bleeding associated with hemophilic patients, who are deficient in key clotting factors (Moll and White, Curr. Opin. Hematol. 2:386-394, 1995; Valentino and Scheiflinger, Semin. Thromb. Hemost. 32(Suppl 2):32-38, 2006).

While the activation of platelets and the coagulation cascade are essential for normal hemostasis in the wound, thrombus formation is a pathological event that may lead to diseases, including myocardial infarction, pulmonary embolism, or stroke, which are the leading causes of death and disability in industrialized nations (Gawaz et al. J. Clin. Invest. 115:3378-3384, 2005; Z. M. Ruggeri, Nat. Med. 8:1227-1234, 2002). To combat the pathologic aggregation of activated platelets, anti-platelet and anticoagulation agents, such as aspirin, warfarin, and heparin, are routinely administered to patients who are considered to be at high, long-term risk of thrombotic disease, while α_{IIb}β₃-blockers are used in acute situations in the clinic (S. A. Mousa, Curr. Pharm. Des. 9:2317-2322, 2003; Phillips et al. J. Thromb. Haemost. 3:1577-1589, 2005). Further, anticoagulants and thrombolytic agents (such as heparins and tissue-type plasminogen activator) are used to prevent or to break up thrombi in various diseases, including ischemic stroke (Albers et al. Chest 126:483 S-512S, 2004; Hacke et al. JAMA 274:1017-1025, 1995).

Protein C(PC) is a serine protease that circulates in the plasma as a zymogen. Human protein C is produced in the liver as a single chain precursor polypeptide of 461 amino acids. Following a series of post-translational modifications, the protein C zymogen is activated by proteolytic cleavage (mediated by thrombin) to produce activated protein C (APC). The activated form of protein C is an anticoagulant. The anticoagulant activity exhibited by APC is a result of its capacity to proteolytically inactivate coagulation factors FVIIIa and FVa, which leads to the inhibition of other components required for blood coagulation, including Factor X and prothrombin.

Although current anti-thrombotic agents are useful in thrombosis by reducing platelet aggregation and clot formation, or by removing thrombi from the circulation, such agents carry deleterious side effects. Systemic anticoagulants reduce fibrin formation and platelet activation in the hemostatic plug and can have severe hemorrhagic side effects, including ischemic stroke. Plasminogen activator treatment, currently the only approved therapy for ischemic stroke in the USA, has been shown to increase the risk of brain hemorrhage, has only a three hour time window of efficacy, and is capable of directly damaging neurons (Benchenane et al. Trends Neurosci. 27:155-160, 2004) Similarly, the same properties of aspirin that lower the clotting action of platelets also cause bleeding (Gorelick and Weisman, Stroke 36:1801-1807, 2005; M. B. Kimmey, Am. J. Med. 117(Suppl 5A):72S-78S, 2005); and oral α_{IIb}β₃-blockers cause a paradoxical increase in cardiovascular disease mortality (Chew et al. Circulation 103:201-206, 2001; Cox et al. J. Am. Coll. Cardiol. 36:1514-1519, 2000; Holmes et al. Am. J. Cardiol. 85:491-493, A410, 2000; Quinn et al. Arterioscler. Thromb. Vasc. Biol. 23:945-952, 2003; Quinn et al. Circulation 106:379-385, 2002; Topol et al. Circulation 108:399-406, 2003), presumably by inadvertently initiating the coagulation cascade. Thus, improved therapies for ischemic/thrombotic events are needed.

Hemostatic agents that minimize bleeding by promoting clotting, such as FEIBA HT™ (activated prothrombin complex concentrate) and NOVOSEVEN™ (Factor VIIa; U.S. Pat. No. 4,784,950), can increase the risk of thrombosis (Turecek et al. Curr. Hematol. Rep. 3(5):331-337, 2004; Levi and Buller, Crit. Care Med. 33(4):883-890, 2005). Therefore, safe hemostatic agents that can correct hemostatic abnormalities, without promotion of thrombus formation, are desirable.

### SUMMARY

This disclosure concerns the surprising finding that protein C polypeptides are effective hemostatic agents. Unlike its activated form, protein C is able to attract platelets and promote localized clotting, while avoiding extension of the clot and formation of a thrombus. Thus, provided herein is a method of promoting hemostasis in a subject by administration of a protein C polypeptide, or a nucleic acid molecule encoding a protein C polypeptide. In some embodiments, the subject has been diagnosed with a bleeding disorder or a bleeding episode. Further provided is a method of preventing, treating or ameliorating a bleeding disorder or a bleeding episode in a subject, comprising administering to the subject a protein C polypeptide, or a nucleic acid molecule encoding a protein C polypeptide. Protein C polypeptides include hemostatic fragments or variants of the protein C polypeptides described herein and known in the art.

The foregoing and other features and advantages will become more apparent from the following detailed description of several embodiments, which proceeds with reference to the accompanying figures.

### BRIEF DESCRIPTION OF THE FIGURES

FIG. 1 is a schematic of the coagulation cascade. Activation of coagulation enzymes leads to the generation of thrombin. An autocatalytic feedback mechanism is propagated by FVIIIa and FVa, while generation of activated protein C (APC) serves to regulate thrombin production.

FIG. 2 is a schematic of cellular platelet receptors and signaling cascades. Receptor-mediated signaling leads to platelet activation. The mechanisms mediating platelet-APC and platelet-protein C(PC) binding are currently undefined.

FIG. 3 shows a hypothetical schematic model of PC-platelet interactions. Platelet GPIb-mediated binding of PC augments ADP release which contributes to platelet activation and thrombus stability (pro-hemostatic activity). Platelet receptors facilitate the activation of PC by thrombin (IIa), leading to localized generation of APC. As an anticoagulant, APC cleaves activated cofactors FVa and FVIIIa (anticoagulant activity).

FIG. 4 is a series of digital images demonstrating platelet spreading and filopodia formation. FIG. 4A shows real-time platelet spreading on immobilized PC, APC, thrombin or fibrinogen at 0, 60, 120, 180, 240, 300 and 510 seconds following platelet addition. FIG. 4B is a series of images of fluorescent-phalloidin staining to show actin stress fibers of platelets on immobilized PC, APC, thrombin or fibrinogen.

FIG. 5 is a series of graphs showing intracellular Ca²⁺elevation of platelets adhered to PC, APC, thrombin (THR) or fibrinogen (FG).

FIG. 6 is a series of digital images of purified human platelets exposed to immobilized ligands (protein C, APC, thrombin or fibrinogen) in the presence of vehicle (−), function-blocking antibody (anti-α_{IIb}β₃) or pharmacological pathway inhibitors (ADP/TxA₂ inhibitors, Src kinase inhibitor, intracellular Ca²⁺chelator). Adherent platelets were fixed and imaged by DIC microscopy.

FIG. 7 is a series of digital images showing platelet adhesion on immobilized PC, APC, thrombin or fibrinogen under shear. Reconstituted blood was perfused over the immobilized ligands at a shear rate of 300⁻¹ for 3 minutes. Platelets were either untreated or treated with an anti-GPIb mAb (10 ₗ μg/ml 6D1), an anti-α_{IIb}β₃mAb (20 μg/ml eptifibatide), or ADP/TxA₂ inhibitors (2 U/ml apyrase; 10 μM indomethacin).

FIG. 8A and FIG. 8B are graphs demonstrating the effect of PC on bleeding time (A) and volume (B) in untreated mice (−) and tPA-treated mice.

FIG. 9 shows a representative data trace for a typical interaction between a PC-bead and an immobilized platelet, partitioned into four parts. The PC-bead, trapped near the center of the laser beam, is moved toward (Upper A) or away (Upper D) from the immobilized platelet, corresponding to zero force (Lower A,D). At the moment of contact (Upper B), the platelet stops the motion of the PC-bead while the laser beam continues in the same direction (right).

FIG. 10A and FIG. 10B are digital images and a graph, respectively, illustrating PC recruitment to immobilized platelets under flow. Platelets were immobilized onto a glass slide treated with 3-aminopropyltriethoxysilane (APES), followed by treatment with 1% BSA. PC or BSA was immobilized onto the surface of 10 μm-diameter polystyrene beads and perfused over the immobilized platelets at a shear rate of 150 s⁻¹ for 5 minutes, in the presence or absence of GPIb antibody.

FIG. 11 is a graph showing platelet-dependent APC generation. A platelet suspension was incubated with PC in the presence of thrombin (Thr) for 60 or 120 minutes. Levels of APC were determined using an APC-specific mAb in conjunction with a HAPC-1555 enzyme capture assay.

FIG. 12A and FIG. 12B are graphs showing the effect of treatment with PC, tPA, or both on hemostasis. Mice were given a bolus injection of 150 μl of either saline or tPA (2 mg/kg) in the presence of either vehicle or recombinant murine PC (3 mg/kg). Bleeding times (A) and bleeding volumes (B) are shown. No animals were allowed to bleed for more than 20 minutes. Bleeding times that exceeded 20 minutes were recorded as being off-scale (dashed line). Horizontal bars represent the mean bleeding time and volume values for each group of animals (n=10-12). ** P≦0.01 with respect to bleeding volume in the presence of tPA exclusively.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NOs: 1 and 2 are the nucleotide and an amino acid sequences of human protein C deposited under GenBank Accession No. NM_{—}000312 on Jun. 21, 2006.

SEQ ID NOs: 3 and 4 are the nucleotide and amino acid sequences of human protein C deposited under GenBank Accession No. BC034377 on Jul. 8, 2002.

SEQ ID NOs: 5 and 6 are the nucleotide and amino acid sequences of human protein C deposited under GenBank Accession No. K02059 on Apr. 27, 1993.

SEQ ID NOs: 7 and 8 are the nucleotide and amino acid sequences of mouse protein C deposited under GenBank Accession No. D10445 on Apr. 29, 1993.

SEQ ID NOs: 9 and 10 are the nucleotide and amino acid sequences of mouse protein C deposited under GenBank Accession No. NM_{—}001042768 on Aug. 17, 2006.

SEQ ID NOs: 11 and 12 are the nucleotide and amino acid sequences of rat protein C deposited under GenBank Accession No. NM_{—}012803 on Feb. 16, 2000.

SEQ ID NOs: 13 and 14 are the nucleotide and amino acid sequences of bovine protein C deposited under GenBank Accession No. XM_{—}585990 on Dec. 22, 2006.

SEQ ID NOs: 15 and 16 are the nucleotide and amino acid sequences of porcine protein C deposited under GenBank Accession No. NM_{—}213918 on May 20, 2004.

SEQ ID NOs: 17 and 18 are the nucleotide and amino acid sequences of a mutant form of protein C.

### DETAILED DESCRIPTION

### I. Abbreviations

ADP Adenosine diphosphate
APC Activated protein C
BSA Bovine serum albumin
ECM Extracellular matrix
FG Fibrinogen
PC Protein C
PRP Platelet-rich plasma
rAPC Recombinant activated protein C
RBC Red blood cell
SDS-PAGE Sodium dodecyl sulfate-polyacrylamide gel electrophoresis
SPR Surface plasmon resonance
TF Tissue factor
tPA Tissue-type plasminogen activator
VWF von Willebrand factor

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Anticoagulant: A compound (such as a pharmaceutical agent or molecule) that prevents or inhibits the clotting of blood. Pharmaceutical anticoagulants can be used to treat thrombotic disorders, such as deep vein thrombosis, pulmonary embolism, myocardial infarction and stroke. The activated form of protein C (APC) is known to function as an anticoagulant.

Bleeding disorder: Refers to any congenital, acquired or induced defect that results in abnormal (or pathological) bleeding. Examples include, but are not limited to, disorders of insufficient clotting or hemostasis, such as hemophilia A (a deficiency in Factor VIII), hemophilia B (a deficiency in Factor IX), hemophilia C (a deficiency in Factor XI), other clotting factor deficiencies (such as Factor VII or Factor XIII), abnormal levels of clotting factor inhibitors, platelet disorders, thrombocytopenia, vitamin K deficiency and von Willebrand's disease.

Bleeding episode: Refers to an occurrence of uncontrolled, excessive and/or pathological bleeding. Bleeding episodes can result from, for example, drug-induced bleeding (such as bleeding induced by non-steroidal anti-inflammatory drugs or warfarin), anticoagulant overdose or poisoning, aneurysm, blood vessel rupture, surgery and traumatic injury (including, for example, abrasions, contusions, lacerations, incisions or gunshot wounds). Bleeding episodes can also result from diseases such as cancer, gastrointestinal ulceration or from infection.

Fusion protein: A protein generated by expression of a nucleic acid sequence engineered from nucleic acid sequences encoding at least a portion of two different (heterologous) proteins. To create a fusion protein, the nucleic acid sequences must be in the same reading frame and contain no internal stop codons.

Hemostasis: Refers to the physiologic process whereby bleeding is halted. Hemostatic agents are those that prevent, treat or ameliorate abnormal bleeding, such as abnormal bleeding caused by a bleeding disorder or bleeding episode. Disorders of hemostasis include, for example, platelet disorders, such as idiopathic thrombocytopenic purpura, and disorders of coagulation, such as hemophilia. Hemostasis can also refer to the complex interaction between vessels, platelets, coagulation factors, coagulation inhibitors and fibrinolytic proteins to maintain the blood within the vascular compartment in a fluid state. The objective of the hemostatic system is to preserve intravascular integrity by achieving a balance between hemorrhage and thrombosis. As described herein, protein C polypeptides and polynucleotides promote hemostasis and are thus useful as hemostatic agents. As used herein, “promoting hemostasis” refers to the process of contributing to or improving hemostasis in a subject. For example, an agent that promotes hemostasis can be an agent that reduces abnormal bleeding, such as by halting bleeding more rapidly, or by reducing the amount of blood loss.

Isolated: An “isolated” biological component (such as a nucleic acid molecule or protein) has been substantially separated or purified away from other biological components of the cell or organism in which the component naturally occurs, such as other chromosomal and extra-chromosomal DNA and RNA, or proteins. Nucleic acids and proteins that have been “isolated” include nucleic acids and proteins purified by standard purification methods. The term also embraces nucleic acids and proteins prepared by recombinant expression in a host cell, as well as chemically synthesized nucleic acids or proteins, or fragments or variants thereof.

Operably linked: A first nucleic acid sequence is operably linked to a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Parenteral: Administered outside of the intestine, for example, not via the alimentary tract. Generally, parenteral formulations are those that will be administered through any possible mode except ingestion. This term especially refers to injections, whether administered intravenously, intrathecally, intramuscularly, intraperitoneally, or subcutaneously, and various surface applications including intranasal, intradermal, and topical application, for instance.

Pharmaceutical agent: A chemical compound or other composition capable of inducing a desired therapeutic or prophylactic effect when properly administered to a subject. Also referred to as a “drug.”

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds or molecules, such as one or more protein C polypeptides or polynucleotides, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide: A polymer in which the monomers are amino acid residues which are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced.

The term “residue” or “amino acid residue” includes reference to an amino acid that is incorporated into a protein, polypeptide, or peptide.

Conservative amino acid substitutions are those substitutions that, when made, least interfere with the properties of the original protein, that is, the structure and especially the function of the protein is conserved and not significantly changed by such substitutions. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

Preventing, treating or ameliorating a disease: “Preventing” a disease refers to inhibiting the full development of a disease. “Treating” refers to a therapeutic intervention that ameliorates one or more signs or symptoms of a disease or pathological condition after it has begun to develop. “Ameliorating” refers to the reduction in the number, duration or severity of signs and/or symptoms of a disease.

Promoter: A promoter is an array of nucleic acid control sequences which direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor).

Protein C(PC): A plasma protein produced as a zymogen. The human protein C zymogen is produced in the liver as a 461 amino acid polypeptide. Proteolytic cleavage of protein C by thrombin produces activated protein C (APC), which possesses anti-coagulant and anti-thrombic activity. As disclosed herein, it has been surprisingly discovered that in contrast to APC, the zymogen form of PC has pro-hemostatic activity. The sequences of mammalian protein C polypeptides and polynucleotides are well known in the art, including those set forth herein as SEQ ID NOs 1-18. As used herein, “protein C polypeptide” includes homologs, variants and fragments of protein C that retain hemostatic activity. Such polypeptides are referred to herein as “hemostatic variants and fragments.” Fragments and variants of protein C polypeptides are well known in the art (see, for example, U.S. Pat. Nos. 5,151,268 and 7,226,999; U.S. Patent Application Publication Nos. 2004-0038288, 2005-0176083 and 2006-0204489; and PCT Publication Nos. WO2004/113385 and WO2006/044294, each of which is herein incorporated by reference). In one example, the protein C polypeptide variant is a variant having one or more mutations in the catalytic site, such as the S360A mutant (see, for example, Gale et al. Protein Sci. 6:132-140, 1997, herein incorporated by reference).

Purified: The term “purified” does not require absolute purity; rather, it is intended as a relative term. Thus, for example, a purified polypeptide, protein or other active compound is one that is isolated in whole or in part from naturally associated proteins and other contaminants, in which the polypeptide, protein or other active compound is purified to a measurable degree relative to its naturally occurring state, for example, relative to its purity within a cell extract or chemical synthesis checker. Methods of purifying protein C polypeptides have been described (see, for example, Gale et al. Protein Sci. 6:132-140, 1997, incorporated herein by reference).

In certain embodiments, the term “substantially purified” refers to a polypeptide, protein, or other active compound that has been isolated from a cell, cell culture medium, or other crude preparation and subjected to fractionation to remove various components of the initial preparation, such as proteins, cellular debris, and other components. Such purified preparations can include materials in covalent association with the polypeptide, such as glycoside residues or materials admixed or conjugated with the polypeptide, which may be desired to yield a modified derivative or analog of the polypeptide or produce a combinatorial therapeutic formulation, conjugate, fusion protein or the like. The term purified thus includes such desired products as peptide and protein analogs or mimetics or other biologically active compounds wherein additional compounds or moieties are bound to the polypeptide in order to allow for the attachment of other compounds and/or provide for formulations useful in therapeutic treatment or diagnostic procedures.

Generally, substantially purified polypeptides, proteins, or other active compounds include more than 80% of all macromolecular species present in a preparation prior to admixture or formulation of the respective compound with additional ingredients in a complete pharmaceutical formulation for therapeutic administration. Additional ingredients can include a pharmaceutical carrier, excipient, buffer, absorption enhancing agent, stabilizer, preservative, adjuvant or other like co-ingredients. More typically, the polypeptide, protein or other active compound is purified to represent greater than 90%, often greater than 95% of all macromolecular species present in a purified preparation prior to admixture with other formulation ingredients. In other cases, the purified preparation can be essentially homogeneous, wherein other macromolecular species are less than 1%.

Recombinant: A recombinant nucleic acid or polypeptide is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, for example, by genetic engineering techniques.

Reporter gene: A reporter gene is a gene operably linked to another gene or nucleic acid sequence of interest (such as a promoter sequence). Reporter genes are used to determine whether the gene or nucleic acid of interest is expressed in a cell or has been activated in a cell. Reporter genes typically have easily identifiable characteristics, such as fluorescence, or easily assayed products, such as an enzyme. Reporter genes can also confer antibiotic resistance to a host cell.

Sequence identity: The similarity between amino acid or nucleotide sequences is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids Research 16:10881-10890, 1988; and Pearson and Lipman, Proc. Natl. Acad. Sci. U.S.A. 85:2444, 1988. Altschul et al., Nature Genet. 6:119-129, 1994.

The NCBI Basic Local Alignment Search Tool (BLAST™) (Altschul et al., J. Mol. Biol. 215:403-410, 1990.) is available from several sources, including the National Center for Biotechnology Information (NCBI, Bethesda, Md.) and on the Internet, for use in connection with the sequence analysis programs blastp, blastn, blastx, tblastn and tblastx.

Variants and/or fragments of protein C generally comprise at least about 80%, at least about 85%, at least about 90%, at least about 95% or at least about 99% sequence identity with a protein C sequence, such as those described herein or described in the art. When less than the entire sequence is being compared for sequence identity, fragments will typically possess at least 80% sequence identity over the length of the fragment, and can possess sequence identities of at least 85%, 90%, 95% or 99%. One of skill in the art will appreciate that these sequence identity ranges are provided for guidance only; it is entirely possible that strongly significant homologs could be obtained that fall outside of the ranges provided.

Subject: Living multi-cellular vertebrate organisms, a category that includes both human and non-human mammals.

Therapeutically effective amount: A quantity of a specified pharmaceutical agent (such as a protein C polypeptide or polynucleotide) sufficient to achieve a desired effect in a subject being treated with the pharmaceutical agent. For example, this may be the amount of a protein C polypeptide useful for preventing, ameliorating, and/or treating a bleeding disorder. When used for prevention, the quantity of agent administered can also be referred to as a “prophylactically effective amount.” Ideally, a therapeutically effective amount (or a prophylactically effective amount) of a pharmaceutical agent is an amount sufficient to promote hemostasis in a subject with a bleeding disorder or bleeding episode, or susceptible to a bleeding disorder or bleeding disorder, without undesired side effects. The effective amount of the pharmaceutical agent will be dependent on the subject being treated, the severity of the affliction, and the manner of administration of the therapeutic composition. For example, a therapeutically effective amount of an active ingredient can be measured as the concentration (moles per liter or molar-M) of the active ingredient in blood (in vivo) or a buffer (in vitro) that produces an effect. An effective amount of a compound can be administered in a single dose, or in several doses, for example daily, during a course of treatment.

Thrombosis: The formation or presence of a clot (also called a “thrombus”) inside a blood vessel, obstructing the flow of blood through the circulatory system. Thrombosis is usually caused by abnormalities in the composition of the blood, quality of the vessel wall and/or nature of the blood flow. The formation of a clot is often caused by an injury to the vessel wall (such as from trauma or infection) and by the slowing or stagnation of blood flow past the point of injury. In some cases, abnormalities in coagulation cause thrombosis.

Transformed: A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Trauma: As used herein, “trauma” or “traumatic injury” refers to a physical injury or wound to the body.

Variants, fragments or fusions: The disclosed protein C polypeptides (such as those set forth as SEQ ID NOs: 2, 4, 6, 8, 10, 12, 14, 16 or 18), include homologs, variants, fragments, and fusions thereof that retain protein C biological activity (such as its pro-hemostatic activity). In some embodiments, a variant or fragment of protein C comprises at least 80% sequence identity with a mammalian protein C polypeptide described herein and/or known in the art. Variants and fragments of protein C are well known in the art, including those described in U.S. Pat. Nos. 5,151,268 and 7,226,999; U.S. Patent Application Publication Nos. 2004-0038288, 2005-0176083; 2006-0204489; and 2008-0658265; and PCT Publication Nos. WO2004/113385; WO2006/044294 and WO 2008/055145, each of which is herein incorporated by reference. In one embodiment, the protein C polypeptide variant is the S360A mutant (see Gale et al. Protein Sci. 6:132-140, 1997 and WO 2008/055145, which are incorporated herein by reference), which contains a serine to alanine change in the catalytic site of the activated enzyme. DNA sequences which encode for a polypeptide or fusion protein thereof, or a fragment or variant of thereof, can be engineered to allow the protein to be expressed in eukaryotic or prokaryotic cells, such as mammalian cells, bacterial cells, insect cells, and plant cells. To obtain expression, the DNA sequence can be altered and operably linked to other regulatory sequences. The final product, which contains the regulatory sequences and the protein of interest, is referred to as a vector. This vector can be introduced into the desired cell. Once inside the cell the vector allows the protein to be produced. One of ordinary skill in the art will appreciate that the DNA can be altered in numerous ways without affecting the biological activity of the encoded protein. For example, PCR can be used to produce variations in the DNA sequence that encodes a protein. Such variants can be variants optimized for codon preference in a host cell used to express the protein, or other sequence changes that facilitate expression.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview of Several Embodiments

Protein C is a plasma protein that exists as a zymogen until proteolytically cleaved by thrombin to produce activated protein C (APC). Through its serine protease activity, APC inactivates the coagulation factors Factor Va and Factor VIIIa, leading to an inhibition of clotting. The anticoagulant activity of APC is important for preventing excessive thrombus formation, which can lead to potentially fatal ischemic events, including stroke or myocardial infarction.

As described herein, it has been surprisingly discovered that protein C, in contrast to its activated form, exhibits hemostatic activity while retaining anti-thrombic activity. Thus, provided herein is a method of promoting hemostasis in a subject, comprising administering to the subject a protein C polypeptide, or a hemostatic fragment or variant thereof. In some embodiments, the subject has been diagnosed with a bleeding disorder or a bleeding episode, and the protein C polypeptide or a hemostatic fragment or variant thereof is administered in a dose that therapeutically improves the bleeding disorder or bleeding episode. Further provided is a method of treating, preventing or ameliorating a bleeding disorder or bleeding episode in a subject, comprising administering to the subject a therapeutically effective amount of a protein C polypeptide, or a hemostatic fragment or variant thereof.

The protein C polypeptides described herein are mammalian protein C polypeptides, including human, mouse, rat, bovine and porcine protein C polypeptides. In one embodiment of the methods, the protein C polypeptide or hemostatic fragment or variant thereof comprises at least 80%, at least 85%, at least 90%, at least 95% or at least 99% sequence identity with the amino acid sequence set forth as SEQ ID NO: 2, 4, 6, 8, 10, 12, 14, 16 or 18. In another embodiment, the protein C polypeptide comprises the amino acid sequence set forth as SEQ ID NO: 2, 4, 6, 8, 10, 12, 14, 16 or 18. In another embodiment, the protein C polypeptide consists of the amino acid sequence set forth as SEQ ID NO: 2, 4, 6, 8, 10, 12, 14, 16 or 18. In some embodiments, the protein C polypeptide is a fragment of protein C, or a variant of protein C, that retains its hemostatic activity (referred to herein as a “hemostatic fragment” or a “hemostatic variant” of protein C). In some embodiments, the protein C fragment comprises at least 50, at least 100, at least 200, at least 300 or at least 400 consecutive amino acids of a protein C polypeptide, such as a protein C polypeptide described herein or known in the art. Fragments and variants of protein C polypeptides are well known in the art. In some embodiments, the protein C polypeptide comprises one or more mutations in an active site of the enzyme. In one example, the protein C polypeptide variant is a protein C polypeptide comprising a mutation in the catalytic site, such as the S360A mutant. In another example, the protein C polypeptide variant comprises the amino acid sequence set forth as SEQ ID NO: 18.

The method of delivery of the polypeptide depends upon, in part, the bleeding disorder or bleeding episode being treated. In some embodiments, the protein C polypeptide or hemostatic fragment or variant thereof is administered by a parenteral route. In one embodiment, the protein C polypeptide or hemostatic fragment or variant thereof is administered intravenously, such as by bolus injection or infusion. In another embodiment, the protein C polypeptide or hemostatic fragment or variant thereof is administered topically, for example, in a gel or ointment, or in a solid form similar to a styptic application in which the hemostatic agent is dissolved by the blood to act at a site of injury. In some cases, topical administration comprises administering the protein C polypeptide as part of a wound dressing. For example, the actual agent can be applied to a bandage or a bioadhesive, such as described in U.S. Pat. Nos. 7,019,191; 7,022,125; 7,196,054; 7,211,651; and 7,230,154, each of which is herein incorporated by reference.

The dose of protein C polypeptide can vary depending upon a variety of factors, including the bleeding disorder or bleeding episode being treated and the subject being treated. A suitable dose can be determined by one of ordinary skill in the art. In some embodiments, the dose of protein C polypeptide, or hemostatic fragment or variant thereof, is about 0.1 mg/day to about 500 mg/day, such as about 0.5 mg/day, about 1.0 mg/day, about 2.5 mg/day, about 5.0 mg/day, about 10 mg/day, about 25 mg/day, about 50 mg/day, about 100 mg/day, about 200 mg/day, about 250 mg/day, about 300 mg/day, about 400 mg/day or about 500 mg/day. In one embodiment, the dose of the protein C polypeptide or hemostatic fragment or variant thereof is about 0.1 to 10 mg/day, such as about 0.1, about 0.2, about 0.5, about 1.0, about 2.5, about 5.0, about 7.5 or about 10 mg/day. In one embodiment, the dose is about 1 to about 100 mg/day. The dose of protein C can also be determined based on the weight of a subject to be treated. Thus, in some embodiments, the dose of a protein C polypeptide is about 0.1 mg/kg to about 10 mg/kg, such as about 0.1 mg/kg, about 0.2 mg/kg, about 0.5 mg/kg, about 1.0 mg/kg, about 2.5 mg/kg, about 5.0 mg/kg, about 7.5 mg/kg, or about 10 mg/kg. In one embodiment, the dose is about 1 to about 10 mg/kg.

The dosing schedule can also vary. In one embodiment, the protein C polypeptide or hemostatic fragment or variant thereof is administered in a single dose. In another embodiment, the protein C polypeptide or hemostatic fragment or variant thereof is administered in multiple doses. The timing of administration can vary depending the bleeding episode or disorder being treated. For example, a patient with a chronic bleeding disorder can be treated regularly, such as twice a day, once a day, twice a week, once a week, twice a month or once a month, or any other appropriate schedule necessary to control bleeding. In another example, a patient preparing to undergo surgery can be treated with protein C prior to surgery, as well as after surgery as needed.

Further provided herein is a method of promoting hemostasis in a subject, comprising administering to the subject a vector comprising a protein C nucleic acid sequence, wherein the protein C nucleic acid sequence encodes a protein C polypeptide or a hemostatic fragment or variant thereof. Also provided is a method of treating, preventing or ameliorating a bleeding disorder or bleeding episode in a subject, comprising administering to the subject a therapeutically effective amount of a vector comprising a protein C nucleic acid sequence, wherein the protein C nucleic acid sequence encodes a protein C polypeptide or a hemostatic fragment or variant thereof.

In some embodiments, the protein C nucleic acid sequence comprises at least 80%, at least 85%, at least 90%, at least 95% or at least 99% sequence identity with the nucleotide sequence set forth as SEQ ID NO: 1, 3, 5, 7, 9, 11, 13, 15 or 17. In another embodiment, the protein C nucleic acid sequence comprises SEQ ID NO: 1, 3, 5, 7, 9, 11, 13, 15 or 17. In another embodiment, the protein C nucleic acid sequence consists of SEQ ID NO: 1, 3, 5, 7, 9, 11, 13, 15 or 17. In other embodiments, the protein C nucleic acid sequence encodes a variant or fragment of protein C that retains its hemostatic activity. In one embodiment, the protein C polynucleotide encoding a protein C variant comprises the nucleic acid sequence set forth as SEQ ID NO: 17. In another embodiment, the protein C nucleic sequence encodes a protein C variant comprising a mutation in the catalytic site, such as the S360A mutant.

In some embodiments, the vector is a viral vector, such as, but not limited to an adenoviral vector, an adeno-associated viral vector, a herpesviral vector, a retroviral vector or a lentiviral vector. In another embodiment, the vector is a eukaryotic expression vector.

In some embodiments, the vector is administered by a parenteral route, such as intravenously. In one embodiment, the vector is administered in a single dose. In another embodiment, the vector is administered in multiple doses.

A protein C polypeptide or polynucleotide can be used as a hemostatic agent for any one of a number of different bleeding disorders or bleeding episodes. In some embodiments, the bleeding disorder is associated with insufficient hemostasis, such as a clotting factor deficiency, a platelet disorder, thrombocytopenia, vitamin K deficiency or von Willebrand's disease. In one embodiment, the clotting factor deficiency is hemophilia A. In another embodiment, the clotting factor deficiency is hemophilia B. In another embodiment, the clotting factor deficiency is hemophilia C. In some embodiments, the bleeding episode is caused by a drug, an anticoagulant overdose, an aneurysm, blood vessel rupture, surgery, a traumatic injury, cancer, gastrointestinal bleeding (from ulceration or trauma) or an infection. In one embodiment, the drug is a non-steroidal anti-inflammatory drug. In another embodiment, the drug is warfarin.

In some cases, a protein C polypeptide or polynucleotide is administered in combination with other pharmaceutical agents that treat bleeding disorders and bleeding episodes by promoting hemostasis. Such pharmaceutical agents include, but are not limited to fibrinogen, thrombin, Factor IX, Factor VII, Factor DC, Factor X, Factor XI, Factor XII, Factor XIII, or activated forms thereof. In addition, protein C polypeptides and polynucleotides can be administered in conjunction with other methods of controlling bleeding and promoting hemostasis, such as surgery, stitches, staples or cauterization.

### IV. Role of Platelets, Thrombin and Protein C in Thrombus Formation

The primary function of platelets is to arrest bleeding. This process requires an orchestrated series of receptor-mediated events facilitating platelet adhesion, rapid cellular activation, and the subsequent accumulation of fibrin and additional platelets into a growing hemostatic plug (Watson et al. J. Thromb. Haemost. 3:1752-1762, 2005). Initial platelet deposition is triggered by the denuding of the endothelium, resulting in the exposure of ECM proteins. ECM-bound von Willebrand factor (VWF) plays a critical role in the tethering of platelets at high shear levels due to the rapid on-rate of binding between GPIb and VWF (McCarty et al. J. Thromb. Haemost. 4:1367-1378, 2006). The rapid off-rate of GPIb-VWF interactions results in platelet translocation at the site of injury, allowing adhesive interactions of receptors with slower binding kinetics (such as integrins) to mediate the firm adhesion of platelets (Jurk and Kehrel, Semin. Thromb. Hemost. 31:381-392, 2005).

Ultimately, these receptor-mediated interactions result in platelet activation, which in turn leads to a rapid remodeling of the actin cytoskeleton, the release of ADP and thromboxanes, and a negatively charged platelet surface (Watson et al. J. Thromb. Haemost. 3:1752-1762, 2005; J. M. Gibbins, J. Cell Sci. 117:3415-3425, 2004; Jackson et al. J. Thromb. Haemost. 1:1602-1612, 2003).

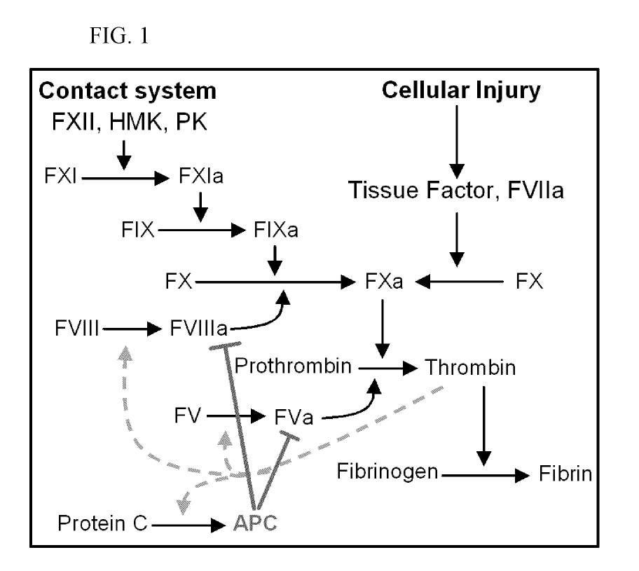

Following vascular injury, concomitant with platelet recruitment and activation, are the first steps of blood coagulation, namely the release of tissue factor (see FIG. 1) (Renne et al. Blood Cells Mol. Dis. 36:148-151, 2006; Renne et al. J. Exp. Med. 202:271-281, 2005; Steffel et al. Circulation 113:722-731, 2006). This process leads to the sequential conversion of other coagulation factors into their corresponding active forms as serine proteases. Protease activation culminates with the generation of thrombin. In the absence of thrombin, hemostatic plugs cannot form (S.R. Coughlin, J. Thromb. Haemost. 3:1800-1814, 2005; Mangin et al. Blood 107:4346-4353, 2006; Sambrano et al. Nature 413:74-78, 2001). Thrombin not only attracts and activates platelets, and cleaves fibrinogen (which leads to fibrin production and clot formation), but thrombin also mediates the feedback activation of the coagulation cofactors, Factor V (FV), VIII (FVIII) and XI (FXI) (Adams and Huntington, Arterioscler. Thromb. Vasc. Biol. 26:1738-1745, 2006). This feedback mechanism leads to an autocatalytic cascade, resulting in rampant clot formation. The essential proenzymes in the hemostatic coagulation process (such as factors VII, IX, X, and prothrombin) require post-translational vitamin K-dependent gamma carboxylation of 9-12 glutamic acid residues on the amino terminus (Gla domain) for proper function (J. Stenflo, Crit. Rev. Eukaryot. Gene Expr. 9:59-88, 1999). The two anticoagulant proteins, PC and protein S, also contain Gla domains (Dahlback and Villoutreix, J. Thromb. Haemost. 1:1525-1534, 2003; D. W. Stafford, J. Thromb. Haemost. 3:1873-1878, 2005).

The anticoagulant effects of PC are due to its activation by thrombin, which results in APC, through the cleavage and release of the PC activation peptide (Griffin et al. Blood Cells Mol. Dis. 36:211-216, 2006). This reaction is slowly catalyzed by thrombin in solution, but the binding of thrombin to its endothelial cell cofactor, thrombomodulin, results in a greater than thousand-fold enhancement of the rate of PC activation (Esmon and Owen, J. Thromb. Haemost. 2:209-213, 2004). The activation of PC is further enhanced (approximately 20-fold) by its binding to the endothelial cell PC receptor on the endothelium (C. T. Esmon, Crit. Care Med. 32:S298-301, 2004). Thus, the generation of APC is restricted to the endothelial cell surface in the current paradigm. As an anticoagulant enzyme, APC with the anticoagulant cofactor, protein S, degrades factors Va and VIIIa, which are required to sustain thrombin formation via the coagulation cascade (FIG. 1). Furthermore, APC cleavage of the endothelial PAR-1 receptor leads to the activation of intracellular G-proteins and the generation of cell protective (anti-apoptotic) responses (D. W. Stafford, J. Thromb. Haemost. 3:1873-1878, 2005). Thus, the coagulation cascade is believed to depend on a delicate balance between pro- and anticoagulant pathways. However, it is unknown whether catalysis of the anticoagulant process is restricted to the endothelium. The notion of restricted generation of the anticoagulant APC on the endothelial cell surface does not take into account the hemodynamics involved in the transport of APC, in whole blood, from the endothelium to the leading edge of the thrombus, a distance that may span from several micrometers to several centimeters (J. J. Hathcock, Arterioscler. Thromb. Vasc. Biol. 26:1729-1737, 2006). It is believed that binding of PC and APC to the platelet surface provides a mechanism for local deactivation of the coagulation cascade.

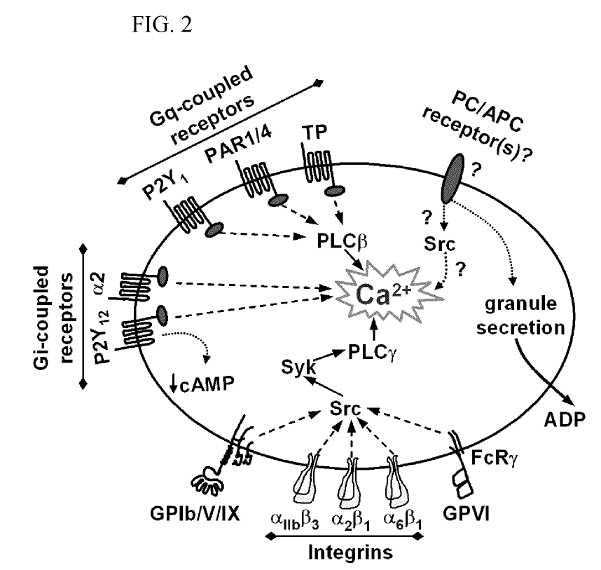

Stimuli originating from agonists released or generated at a site of vascular injury act via signaling networks to enhance (within seconds) the adhesive and pro-coagulant properties of platelets. As shown in FIG. 2, the platelet receptors α_{IIb}β₃ and GPVI, which bind fibrinogen and collagen, respectively, have been shown to induce platelet activation through a pathway that is dependent on the Src family and Syk tyrosine kinases, and on the activation of the effector enzyme PLCγ2, leading to a dramatic rise in cytosolic calcium flux (J. M. Gibbins, J. Cell Sci. 117:3415-3425, 2004; Jackson et al. J. Thromb. Haemost. 1:1602-1612, 2003). The G protein-coupled receptors PAR-1/4 and P2Y₁, which are activated by thrombin and ADP, respectively, lead to platelet activation in a PLCβ-dependent manner (Lundblad and White, Platelets 16:373-385, 2005; Oury et al. Curr. Pharm. Des. 12:859-875, 2006). A propagation phase follows, whereby platelets secrete mediators such as ADP and thromboxane A₂ (TxA₂), which activate other platelets to form aggregates. In the subsequent perpetuation phase of platelet plug formation, fibrin generation and post-aggregation events are believed to stabilize the thrombus.

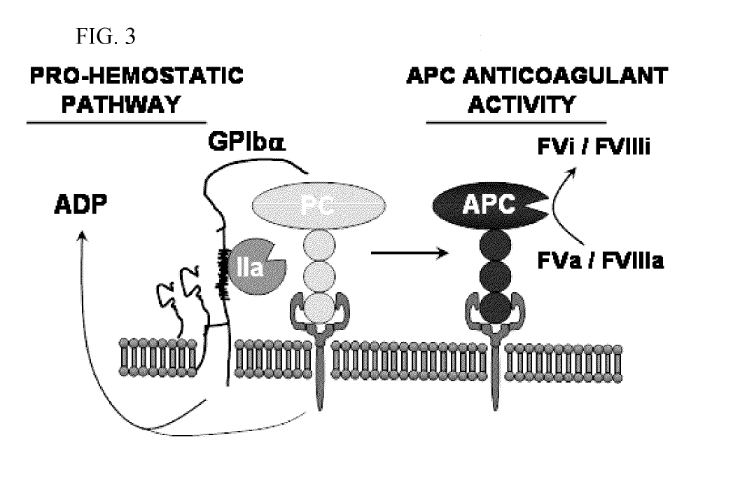

The studies described in the Examples herein indicate that PC and APC binding to platelets induces ADP secretion and that PC- and APC-platelet interactions are pro-hemostatic and contribute to thrombus stability (FIG. 3A). This model is analogous to the scenario described for PC binding to endothelial cells which, upon the conversion of PC to APC, APC stimulates endothelial cell activation through the cleavage of PAR-1 (Feistritzer et al. J. Biol. Chem. 281:20077-20084, 2006). Furthermore, in parallel to the anticoagulant role that APC plays on the endothelial cell surface, the data described in the Examples herein indicates that APC generation on the platelet surface plays a role in down-regulating thrombogenesis in the lumen of blood vessels (FIG. 3B).

### V. Protein C Polypeptides and Polynucleotides

This disclosure provides the surprising finding that administration of a protein C polypeptide promotes hemostasis by reducing bleeding time and blood volume lost. This finding is surprising given the function of APC as an anti-coagulant. The data described herein indicate that protein C is pro-hemostatic and administration of protein C can inhibit (including prevent), treat or ameliorate bleeding in a subject by promoting hemostasis in a subject with a bleeding disorder or bleeding episode. Thus, provided herein are mammalian protein C polypeptides, termed “protein C polypeptides” for use as hemostatic agents. Human protein C amino acid sequences are known in the art, including, but not limited to human protein C deposited under GenBank Accession No. NM_{—}000312 on Jun. 21, 2006 (SEQ ID NO: 2); GenBank Accession No. BC034377 on Jul. 8, 2002 (SEQ ID NO: 4); and GenBank Accession No. K02059 on Apr. 27, 1993 (SEQ ID NO: 6). Mouse protein C sequences, include but are not limited to the protein C sequences deposited under GenBank Accession No. D10445 on Apr. 29, 1993 (SEQ ID NO: 8: and GenBank Accession No. NM_{—}001042768 on Aug. 17, 2006 (SEQ ID NO: 10). Rat, bovine and porcine protein C polypeptides respectively include the sequences deposited under GenBank Accession No. NM_{—}012803 on Feb. 16, 2000 (SEQ ID NO: 12); GenBank Accession No. XM_{—}585990 on Dec. 22, 2006 (SEQ ID NO: 14); and GenBank Accession No. NM_{—}213918 on May 20, 2004 (SEQ ID NO: 16).

Specific, non-limiting examples of protein C polypeptides include variants and/or fragments of protein C polypeptides, including polypeptides having an amino acid sequence at least about 80%, at least about 85%, at least about 90%, at least about 95%, or at least about 99% identical to the amino acid sequence set forth as SEQ ID NO: 2, 4, 6, 8, 10, 12, 14, 16 or 18. In a further embodiment, a protein C polypeptide is a conservative variant of SEQ ID NO: 2, 4, 6, 8, 10, 12, 14, 16 or 18, such that it includes no more than twenty-five conservative amino acid substitutions, such as no more than two, no more than five, no more than ten, no more than fifteen, no more than twenty, or no more than twenty-five conservative amino acid substitutions in SEQ ID NO: 2, 4, 6, 8, 10, 12, 14, 16 or 18. In another embodiment, a protein C polypeptide comprises an amino acid sequence as set forth as SEQ ID NO: 2, 4, 6, 8, 10, 12, 14, 16 or 18. In another embodiment, a protein C polypeptide consists of an amino acid sequence as set forth as SEQ ID NO: 2, 4, 6, 8, 10, 12, 14, 16 or 18.

Fragments and variants of a protein C polypeptide can readily be prepared by one of skill in the art using molecular techniques. In one embodiment, a fragment of a protein C polypeptide includes at least 50, 100, 200, 300 or 400 consecutive amino acids of the protein C polypeptide. In a further embodiment, a fragment or variant of protein C is a fragment or variant that retains hemostatic activity. Such fragments and variants are referred to as “hemostatic fragments and variants.” Fragments and variants of protein C polypeptides are well known in the art (see, for example, U.S. Pat. Nos. 5,151,268 and 7,226,999; U.S. Patent Application Publication Nos. 2004-0038288, 2005-0176083; 2006-0204489; and 2008-0658265; and PCT Publication Nos. WO 2004/113385; WO 2006/044294; and WO 2008/055145, each of which is herein incorporated by reference). In one embodiment, the protein C polypeptide variant comprises the amino acid sequence of SEQ ID NO: 18.

In another embodiment, the protein C polypeptide variant is a protein C polypeptide having a mutation in a catalytic site, such as the S360A mutant (see WO 2008/055145 and Gale et al. Protein Sci. 6:132-140, 1997, herein incorporated by reference). The S360A mutant, when proteolytically processed to form APC, lacks amidolytic activity but retains significant (although not complete) anti-coagulant activity and is not inhibited by serine protease inhibitors. In addition, the S360A mutant has an increased half life in vivo. As shown herein, the S360A mutant of PC also exhibits pro-hemostatic activity, indicating that the hemostatic activity of PC does not require catalytic activity of APC.

One skilled in the art can purify a protein C polypeptide using standard techniques for protein purification. The substantially pure polypeptide will yield a single major band on a non-reducing polyacrylamide gel. The purity of the protein C polypeptide can also be determined by amino-terminal amino acid sequence analysis. Expression and purification of recombinant protein C has been described (Gale et al. Protein Sci. 6:132-140, 1997, incorporated herein by reference).

Minor modifications of the protein C polypeptide primary amino acid sequences may result in peptides which have substantially equivalent activity as compared to the unmodified counterpart polypeptide described herein. Such modifications may be deliberate, as by site-directed mutagenesis, or may be spontaneous. All of the polypeptides produced by these modifications are included herein.

One of skill in the art can readily produce fusion proteins including a protein C polypeptide and a second polypeptide of interest. Optionally, a linker can be included between the protein C polypeptide and the second polypeptide of interest. Fusion proteins include, but are not limited to, a polypeptide including a protein C polypeptide and a marker protein. In one embodiment, the marker protein can be used to identify or purify a protein C polypeptide. Exemplary fusion proteins include, but are not limited to, green fluorescent protein, six histidine residues, or myc, and a protein C polypeptide.

Polynucleotides encoding a mammalian protein C polypeptide are also provided, and are termed “protein C polynucleotides.” These polynucleotides include DNA, cDNA and RNA sequences which encode a mammalian protein C. Exemplary polynucleotide sequences encoding protein C are known in the art, such as, but not limited to human protein C deposited under GenBank Accession No. NM_{—}000312 on Jun. 21, 2006 (SEQ ID NO: 1); GenBank Accession No. BC034377 on Jul. 8, 2002 (SEQ ID NO: 3); and GenBank Accession No. K02059 on Apr. 27, 1993 (SEQ ID NO: 5). Mouse protein C nucleic acid sequences, include but are not limited to the protein C sequences deposited under GenBank Accession No. D10445 on Apr. 29, 1993 (SEQ ID NO: 7: and GenBank Accession No. NM_{—}001042768 on Aug. 17, 2006 (SEQ ID NO: 9). Rat, bovine and porcine protein C polynucleotides respectively include the sequences deposited under GenBank Accession No. NM_{—}012803 on Feb. 16, 2000 (SEQ ID NO: 11); GenBank Accession No. XM_{—}585990 on Dec. 22, 2006 (SEQ ID NO: 13); and GenBank Accession No. NM_{—}213918 on May 20, 2004 (SEQ ID NO: 15).

Specific, non-limiting examples of protein C polynucleotides include polynucleotides that encode variants and/or fragments of protein C polypeptides. Such protein C polynucleotides include polynucleotides having a nucleic acid sequence at least about 80%, at least about 85%, at least about 90%, at least about 95%, or at least about 99% identical to the nucleic acid sequence set forth as SEQ ID NO: 1, 3, 5, 7, 9, 11, 13, 15 or 17. In a further embodiment, a protein C polynucleotide encodes a conservative variant of the amino acid sequence set forth as SEQ ID NO: 2, 4, 6, 8, 10, 12, 14, 16 or 18, such that the encoded variant includes no more than twenty-five conservative amino acid substitutions, such as no more than two, no more than five, no more than ten, no more than fifteen, no more than twenty, or no more than twenty-five conservative amino acid substitutions in SEQ ID NO: 2, 4, 6, 8, 10, 12, 14, 16 or 18. In another embodiment, a protein C polynucleotide comprises a nucleic acid sequence as set forth as SEQ ID NO: 1, 3, 5, 7, 9, 11, 13, 15 or 17. In another embodiment, a protein C polynucleotide consists of a nucleic acid sequence as set forth as SEQ ID NO: 1, 3, 5, 7, 9, 11, 13, 15 or 17. In some embodiments, the protein C polynucleotide encodes a protein C polypeptide variant. In one example, the nucleic acid encoding a protein C polypeptide variant comprises SEQ ID NO: 17. In another example, the protein C polynucleotide encodes a protein C polypeptide variant having a mutation in the catalytic site, such as the S360A mutant.

A polynucleotide encoding a mammalian protein C polypeptide can be included in an expression vector to direct expression of the protein C nucleic acid sequence. Other expression control sequences, including appropriate promoters, enhancers, transcription terminators, a start codon, splicing signals for introns, and stop codons can be included in an expression vector. Generally expression control sequences include a promoter, a minimal sequence sufficient to direct transcription. Expression vectors comprising protein C polynucleotide sequences can be used to transform cells for amplification and purification of protein C polypeptide. Vectors encoding protein C polynucleotides also can be used to directly administer to a subject with a bleeding disorder or bleeding episode.

### VI. Vectors and Cells for Expression and Delivery of Protein C

Suitable vectors for expression of protein C will typically contain an origin of replication and a promoter. Vectors can optionally comprise a specific gene which allows for phenotypic selection of the transformed cells (for example, an antibiotic resistance cassette), a marker gene to enable purification of the expressed protein, and/or a reporter gene for detection of expression.

Suitable vectors include, but are not limited to, prokaryotic and eukaryotic expression vectors. Expression vectors are well known in the art (see for example, Eukaryotic Viral Vectors, Cold Spring Harbor Laboratory, Gluzman ed., 1982). Examples of eukaryotic expression vectors include pMSXND (Lee and Nathans, J. Biol. Chem. 263:3521, 1988), and pCIS2M (Gale et al. Protein Sci. 6:132-140, 1997, incorporated herein by reference).

Other suitable vectors include viral vectors. Viral vectors can be used to deliver protein C polynucleotides to cells for amplification and purification of protein C polypeptides, or viral vectors can be used to administer protein C polynucleotides to a subject. Specific, non-limiting examples of viral vectors include, but are not limited to, adenovirus vectors, adeno-associated virus vectors, retroviral vectors, lentiviral vectors, herpesviral vectors, and the like.

Adenovirus vectors can be first, second, third and/or fourth generation adenoviral vectors or gutless adenoviral vectors. Adenovirus vectors can be generated to very high titers of infectious particles; infect a great variety of cells; efficiently transfer genes to cells that are not dividing; and are seldom integrated in the host genome, which avoids the risk of cellular transformation by insertional mutagenesis (Douglas and Curiel, Science and Medicine, March/April 1997, pages 44-53; Zern and Kresinam, Hepatology 25(2), 484-491, 1997). Representative adenoviral vectors which can be used for the methods provided herein are described by Stratford-Perricaudet et al. (J. Clin. Invest. 90: 626-630, 1992); Graham and Prevec (In Methods in Molecular Biology: Gene Transfer and Expression Protocols 7: 109-128, 1991); and Barr et al. (Gene Therapy, 2:151-155, 1995), which are herein incorporated by reference.

Adeno-associated virus (AAV) vectors also are suitable for expression and/or delivery of protein C. Methods of generating AAV vectors, administration of AAV vectors and their use are well known in the art (see, for example, U.S. Pat. No. 6,951,753; U.S. Pre-Grant Publication Nos. 2007-036757, 2006-205079, 2005-163756, 2005-002908; and PCT Publication Nos. WO 2005/116224 and WO 2006/119458, each of which is herein incorporated by reference).

Retrovirus, including lentivirus, vectors can also be used with the methods described herein. Lentiviruses include, but are not limited to, human immunodeficiency virus (such as HIV-1 and HIV-2), feline immunodeficiency virus, equine infectious anemia virus and simian immunodeficiency virus. Other retroviruses include, but are not limited to, human T-lymphotropic virus, simian T-lymphotropic virus, murine leukemia virus, bovine leukemia virus and feline leukemia virus. Methods of generating retrovirus and lentivirus vectors and their uses have been well described in the art (see, for example, U.S. Pat. Nos. 7,211,247; 6,979,568; 7,198,784; 6,783,977; and 4,980,289, each of which is herein incorporated by reference).

Suitable herpesvirus vectors can be derived from any one of a number of different types of herpesviruses, including, but not limited to, herpes simplex virus-1 (HSV-1), HSV-2 and herpesvirus saimiri. Recombinant herpesvirus vectors, their construction and uses are well described in the art (see, for example, U.S. Pat. Nos. 6,951,753; 6,379,6741 6,613,892; 6,692,955; 6,344,445; 6,319,703; and 6,261,552; and U.S. Pre-Grant Publication No. 2003-0083289, each of which is herein incorporated by reference).

Expression vectors for use in expressing protein C polypeptides comprise a promoter capable of directing the transcription of a nucleic acid encoding protein C. Suitable promoters are well known in the art. Promoters for use in cultured mammalian cells include viral promoters and cellular promoters. Viral promoters include the SV40 promoter (Subramani et al. Mol. Cell. Biol. 1:854-864, 1981); the CMV promoter (Boshart et al. Cell 41:521-530, 1985); and the major late promoter from adenovirus 2 (Kaufman and Sharp, Mol. Cell. Biol. 2:1304-1319, 1982). Cellular promoters include the mouse kappa gene promoter (Bergman et al. Proc. Natl. Acad. Sci. U.S.A. 81:7041-7045, 1983); the mouse V_{H} promoter (Loh et al. Cell 33:85-93, 1983); and the mouse metallothionein-I promoter (Palmiter et al. Science 222:809-814, 1983). Other suitable promoters include, but are not limited to, the thymidine kinase promoter (TK) and the beta-actin promoter. The promoter can be inducible or constitutive. The promoter can also be tissue specific.

In one embodiment, the polynucleotide encoding a protein C polypeptide is located downstream of the desired promoter. Optionally, an enhancer element is also included, and can generally be located anywhere on the vector and still have an enhancing effect. However, the amount of increased activity will generally diminish with distance.

Expression vectors can also contain a set of RNA splice sites located downstream from the promoter and upstream from the insertion site for the protein C polynucleotide sequence. Preferred RNA splice sites can be obtained from adenovirus and/or an immunoglobulin gene. Expression vectors can optionally comprise a polyadenylation signal located downstream of the insertion site. Examples of polyadenylation signals include the early or late polyadenylation signal from SV40, the polyadenylation signal from the adenovirus 5 E1b region, or the human growth hormone gene terminator (DeNoto et al. Nucl. Acids Res. 9:3719-3730, 1981).

Expression vectors comprising a polynucleotide encoding mammalian protein C can be used to transform host cells. Hosts can include isolated microbial, yeast, insect and mammalian cells, as well as cells located in an organism. Biologically functional viral and plasmid DNA vectors capable of expression and replication in a host are known in the art, and can be used to transfect any cell of interest. Where the cell is a mammalian cell, the genetic change is generally achieved by introduction of the DNA into the genome of the cell or as an episome. Thus, host cells can be used to produce protein C polypeptides. Alternatively, expression vectors can be used to transform host cells of interest.

A transfected or transformed cell is a cell into which a nucleic acid molecule (such as a nucleic acid molecule encoding a protein C polypeptide) has been introduced by means of recombinant DNA techniques. Transfection of a host cell with recombinant nucleic acid can be carried out by conventional techniques as are well known in the art. Where the host is prokaryotic, such as E. coli, competent cells which are capable of nucleic acid uptake can be prepared from cells harvested after exponential growth phase and subsequently treated by the CaCl₂ method using procedures well known in the art. Alternatively, MgCl₂ or RbC1 can be used. Transformation can also be performed after forming a protoplast of the host cell if desired, or by electroporation. Nucleic acid sequences are introduced into cultured mammalian cells by, for example, calcium phosphate-mediated transfection (Wigler et al. Cell 14:725-732, 1978; Corsaro and Pearson, Somatic Cell Genetics 7:603-616, 1981; Graham and Van der Eb, Virology 52d:456-467, 1973), electroporation (Neumann et al. EMBO J. 1:841-845, 1982), microinjection, liposome-mediated transfection, or by viral vector.

### VII. Administration of Protein C Compositions

The protein C polypeptides and polynucleotides described herein can be used to treat, prevent or ameliorate a bleeding disorder or bleeding episode in a subject in need thereof. In one embodiment, the subject is administered a protein C polypeptide. In another embodiment, the subject is administered a nucleic acid molecule encoding a protein C polypeptide. Such nucleic acid molecules encoding protein C polypeptide can be administered in the form of a vector, such as a viral vector.

Protein C polypeptides and polynucleotides are usually administered to a subject as compositions comprising one or more pharmaceutically acceptable carriers. Such carriers are determined in part by the particular composition being administered, as well as by the particular method used to administer the composition. Accordingly, there is a wide variety of suitable formulations of pharmaceutical compositions of the present disclosure.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like. In one embodiment, the protein C polypeptides or polynucleotides are delivered intravenously in combination with a pharmaceutically acceptable carrier.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable. In one embodiment, the protein C polypeptide is administered topically as part of a wound dressing.

Administration can be accomplished by single or multiple doses. The dose required will vary from subject to subject depending on the species, age, weight, general condition of the subject, the particular bleeding disorder or episode being treated, the particular protein C polypeptide or polynucleotide being used and its mode of administration. An appropriate dose can be determined by one of ordinary skill in the art using only routine experimentation. In some embodiments, the dose of protein C polypeptide is about 0.1 mg/day to about 500 mg/day, such as about 0.5 mg/day, about 1.0 mg/day, about 2.5 mg/day, about 5.0 mg/day, about 10 mg/day, about 25 mg/day, about 50 mg/day, about 100 mg/day, about 200 mg/day, about 250 mg/day, about 300 mg/day, about 400 mg/day or about 500 mg/day. In one embodiment, the dose is about 1 to about 100 mg/day. The dose of protein C can also be determined based on the weight of a subject to be treated. Thus, in some embodiments, the dose of a protein C polypeptide is about 0.1 mg/kg to about 10 mg/kg, such as about 0.1 mg/kg, about 0.2 mg/kg, about 0.5 mg/kg, about 1.0 mg/kg, about 2.5 mg/kg, about 5.0 mg/kg, about 7.5 mg/kg, or about 10 mg/kg. In one embodiment, the dose is about 1 to about 10 mg/kg.

For treating a chronic bleeding disorder, a subject can be administered an appropriate dose of protein C polypeptide or polynucleotide on a regular schedule, such as, for example, weekly, daily or twice daily. To prophylactically treat a patient at risk for developing a bleeding episode (such as a subject scheduled for surgery), the protein C polypeptides or polynucleotides can be administered prior to the bleeding episode (i.e., the surgery), such as 12, 24 or 48 hours prior. In the case of a scheduled surgery, it may be appropriate to administer protein C in a single dose or in multiple doses. In the case of a wound treated with protein C polypeptide topically, the dosing schedule can depend, in part, on the frequency with which the wound dressing is replaced.

Protein C polypeptides and polynucleotides can also be administered in combination with other pharmaceutical agents that treat bleeding disorders and bleeding episodes. Such pharmaceutical agents include, but are not limited to fibrinogen, thrombin, Factor IX, Factor VII, Factor DC, Factor X, Factor XI, Factor XII, Factor XIII, or activated forms thereof. In addition, protein C polypeptides and polynucleotides can be administered in conjunction with other methods of controlling bleeding, such as surgery, stitches, staples or cauterization.

The following examples are provided to illustrate certain particular features and/or embodiments. These examples should not be construed to limit the disclosure to the particular features or embodiments described.

### EXAMPLES

### Example 1

### Methods

### Protein C(PC) and Activated Protein C (APC) Polypeptides

Human plasma derived PC and APC can be purchased from Hematologic Technologies (Essex Junction, Vt.). These PC preparations are free of detectable APC (<0.1%) and thrombin (<0.04 pM in 100 nM PC) as determined by amidolytic assays (Feistritzer et al. J Biol. Chem. 281:20077-20084, 2006).

PC was purified from plasma factor IX concentrate using immunoaffinity chromatography (Gruber et al. Circulation 82:578-585, 1990; Gruber et al. Circulation 84:2454-2462, 1991). Anti-human PC light-chain mAbs designated C3′5 were coupled to CNBr-activated Sepharose 4B (Pharmacia; 3 mg protein/mL gel) in a coupling buffer (0.5 mol/L NaCl, 0.05 mol/L borate, pH 8.5) overnight at 4° C. The Factor IX concentrate was passed over C3-Sepharose in a buffer containing 0.1 mol/L NaCl, 2 mmol/L EDTA, 2 mmol/L benzamidine, 0.02% Na-azide, 0.02% Tween-20, and 0.02 mol/L Tris-HCl, pH 7.4; and subsequently eluted with 3 mol/L NaSCN in 1.0 mol/L NaCl, 4 mmol/L benzamidine, 2 mmol/L EDTA, 0.02% Na-azide, 0.05% Tween-20, and 0.05 mol/L Tris, pH 7.0. This purification process yields PC that is >98% pure when analyzed using sodium dodecyl sulfate-polyacrylamide gel electrophoresis (SDS-PAGE).

To generate APC, PC in tris-buffered saline (TBS) (0.15 mol/L NaCl, 0.01 mol/L Tris, pH 7.4) was activated using thrombin-Sepharose beads. This process generates APC that does not contain any detectable thrombin, as measured by a clotting assay, and is >95% activated according to SDS-PAGE (Gale et al. Protein Sci. 6:132-140, 1997; Gale et al. J. Biol. Chem. 277:28836-28840, 2002).

Mutant PC expression vectors can be constructed, and recombinant PC mutants can be purified from conditioned media (Gale et al. Protein Sci. 6:132-140, 1997; Gale et al. J. Biol. Chem. 277:28836-28840, 2002). Purified PC can be activated by thrombin to form APC. PC in HBS (50 mM HEPES, 150 mM NaCl) with 2 mM EDTA and 0.5% BSA, pH 7.4, at a concentration of 600 μg/mL is incubated for 2.5 hrs with 12 μg/mL thrombin at 37° C. followed by the addition of 1.1 U of hirudin per U of thrombin to inactivate the thrombin. Subsequently, thrombin is removed by anion-exchange chromatography with NaCl gradient elution. Residual thrombin, as determined by fibrin clotting, accounts for <0.00025% (moles of thrombin per moles of APC) of the protein (Mosnier et al. Blood 104:1740-1744, 2004). Concentrations of recombinant wild-type APC and APC mutants are determined by active-site titration using APC at 8 μM in HBS and p-nitrophenol-guanidino benzoate at 0.1 mM and using an extinction coefficient for p-nitrophenol of 11 400 M⁻¹ cm⁻¹ (at pH 7.4) (Gale et al. Protein Sci. 6:132-140, 1997; Mosnier et al. Blood 104:1740-1744, 2004). The concentration of S360A-APC can be determined by Asserachrom PC ELISA from American Bioproducts (Parsippany, N.J.).

### Glycocalicin Purification

Glycocalicin was isolated from outdated human platelets (Simon et al. J. Exp. Med. 192:193-204, 2000; Yun et al. J. Biol. Chem. 278:48112-48119, 2003). Erythrocyte-free platelets were isolated from 10 liters of outdated platelet-rich plasma by centrifugation and washing with buffer A (13 mM Na₃ citrate, 120 mM NaCl, and 30 mM glucose, pH 7.0). After a second centrifugation, the platelet pellet was suspended in 500 ml of buffer B (10 mM Tris/HCl, 150 mM NaCl, and 2 mM CaCl₂, pH 7.4) and then sonicated. The resultant suspension was incubated at 37° C. for 30 minutes to allow the calpain released from the platelets during sonication to cleave membrane-bound GPIbα releasing glycocalicin. After ultracentrifugation to remove cell components, the glycocalicin-containing supernatant was applied to a wheat germ Sepharose 4B column. Bound crude glycocalicin was eluted with 2.5% N-acetyl-D-glucosamine and 20 mM Tris/HCl, pH 7.4. Further purification by ion exchange chromatography (on Q-Sepharose Fast-flow column; Pharmacia) was used to remove residual contaminants. Glycocalicin was eluted with a linear salt gradient of 0-0.7 M NaCl in 20 mM Tris/HCl, pH 7.4.

### Platelet Purification

Human venous blood from healthy volunteers was drawn by venipuncture into sodium citrate and acid/citrate/dextrose (ACD) (McCarty et al. J. Biol. Chem. 280:39474-39484, 2005; McCarty et al. J. Thromb. Haemost. 4:1367-1378, 2006). Platelet-rich plasma (PRP) was prepared by centrifugation of whole blood at 200 g for 20 minutes. Platelets were isolated from PRP by centrifugation at 1000 g for 10 minutes in the presence of prostacyclin (0.1 μg/ml). Platelet pellets were resuspended in Tyrodes containing 0.1 μg/ml prostacyclin, then washed and resuspended in modified Tyrodes buffer (129 NaCl mM, 0.34 mM Na₂HPO₄, 2.9 mM KCl, 12 mM NaHCO₃, 20 mM HEPES, 5 mM glucose, 1 mM MgCl₂; pH 7.3).

For flow adhesion studies using reconstituted blood, autologous RBCs were isolated following the initial centrifugation of whole blood (200 g for 20 minutes). After the removal of PRP, the RBCs were pelleted by further centrifugation (2000 g for 10 min), followed by washing (3×) with a HEPES buffer (10 mM HEPES, 140 mM NaCl, 5 mM glucose). Washed platelets (3×10⁸/ml final) were reconstituted with 50% (v/v) autologous-packed RBCs.

### Hemostasis Test in Mice

C57BL6 mice weighing 21-23 grams were purchased from Charles River Laboratories (Madison, Wis.) and used for hemostasis assessment with the tail transection test. Each animal was euthanized following the bleeding test. Mice were anesthetized with isoflurane and infused for 2 minutes through the right femoral vein with tPA (2 mg/kg, 150 μL, total vehicle volume) to impair hemostasis. The effect of protein C on hemostasis was tested by co-administration of wild-type or active site mutated (S360A) mouse protein C (3 mg/kg) in combination with tPA. Control animals received physiological saline with or without tPA. Anesthesia was terminated immediately after the infusion, and mice were immobilized in a restraining device that allowed free access to the tail. The tail transection bleeding test was performed 20 minutes after the end of tPA/PC or vehicle administration. A disposable surgical blade was used to cut the tail at the point where the tail diameter reached approximately 1.5 mm (2-4 mm from the tip). After transection, the tail was immediately placed in a 1.7 ml tube filled with 500 μl of room temperature water. Bleeding time (by visual observation) and total volume of blood accumulated in the tube until the end of bleeding were recorded for up to 20 minutes.

### Example 2

### Characterization of Platelet Adhesion and Activation on PC

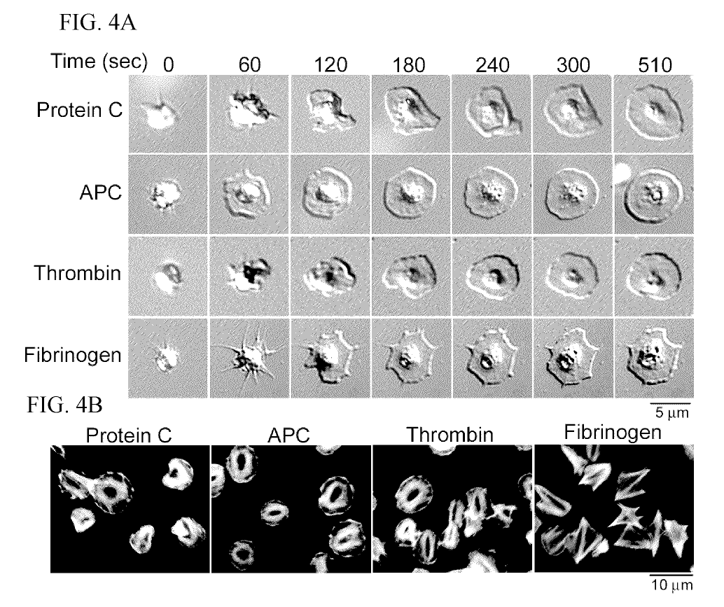

To characterize the ability of platelets to bind PC and APC, purified human platelets were gently pipetted over surface-immobilized PC and APC. The purity of PC and APC was verified by SDS-PAGE. Platelet spreading, which is contingent on platelet activation, was monitored using Normarski DIC microscopy. As shown in FIG. 4A, human platelets underwent complete spreading on PC and APC, and the spreading was characterized by the generation of limited small filopodia followed by wave-like lamellipodia appearing before filopodia formation was complete. Actin stress fiber formation was observed on both PC and APC (FIG. 4B). A similar pattern of platelet spreading and stress fiber formation was observed on thrombin, while a sequential formation of discrete filopodia and lamellipodia was observed on fibrinogen.

To determine whether platelet adhesion to PC or APC resulted from either a receptor-mediated or an agonist-induced mechanism, the effect of PC, APC or thrombin in suspension on platelet binding was evaluated. Purified human platelets (2×10⁷/ml) were placed on BSA, PC, APC, recombinant APC (rAPC), or thrombin-coated coverslips for 45 minutes at 37° C. In designated experiments, washed platelets were resuspended in buffer containing exogenously added PC, APC, rAPC or thrombin (50 μg/ml), or the ADP-scavenger apyrase (apy) and TxA₂ inhibitor indomethacin (indo) (2 U/ml apy; 10 μM indomethacin), prior to exposure to the immobilized protein surface. Adherent platelets are reported as mean±SEM>300 cells from 3-6 experiments. The results demonstrated that the addition of PC and APC in suspension abrogated the ability of platelets to bind to immobilized PC and APC surfaces, respectively (Table 1).

TABLE 1
Effect of ligands in suspension on platelet
binding to immobilized ligands

| | Suspension | Platelet adhesion | Platelet surface |
|---|---|---|---|
| Surface | treatment | (cells/mm² × 10⁻²) | area (μm²) |
| PC | — | 35.4 ± 4.4 | 30.2 ± 1.1 |
| PC | PC | 3.8 ± 1.8* | 9.9 ± 0.2 |
| PC | apy/indo | 11.2 ± 2.0* | 16.2 ± 0.9* |
| APC | — | 50.5 ± 8.5 | 31.8 ± 1.0 |
| APC | APC | 4.2 ± 2.5* | 10.2 ± 0.4* |
| APC | apy/indo | 18.8 ± 6.3* | 15.1 ± 0.8* |
| rAPC | — | 41.8 ± 2.9 | 31.2 ± 1.3 |
| rAPC | rAPC | 2.6 ± 1.2* | 10.0 ± 0.5* |
| thrombin | — | 140.0 ± 9.6 | 39.1 ± 0.6 |
| thrombin | thrombin | 110.8 ± 6.5 | 40.0 ± 0.5 |
| thrombin | apy/indo | 111.4 ± 17.0 | 38.4 ± 0.7 |

*P < 0.01 with respect to untreated samples for each surface

Platelets were found to bind to and spread on recombinant APC (rAPC). This adhesion to rAPC was eliminated when rAPC was present in suspension, indicating that adhesion to APC was unlikely due to contaminating factors, such as fibrinogen or VWF, in the plasma-derived APC. The presence of thrombin in the platelet suspension did not alter the number of platelets binding to immobilized thrombin. Together, these results suggest that platelet binding to PC and to APC is a receptor-mediated, rather than an enzymatic, process as evidenced by the ability of PC, APC, and rAPC in solution to competitively inhibit binding to immobilized ligands.

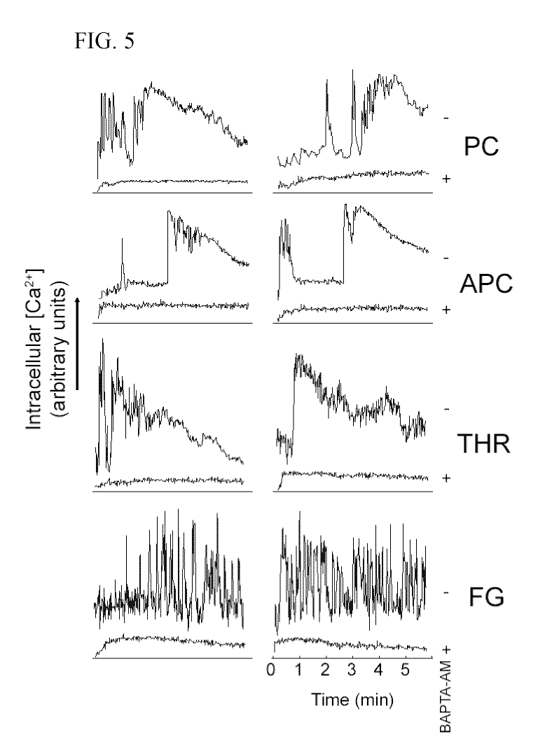

To evaluate the ability of PC and APC to induce outside-in signaling, intracellular Ca²⁺flux of adherent platelets was examined on each surface. Platelets loaded with the Ca²⁺-sensitive dye, Oregon Green BAPTA 1-AM, were pipetted onto each surface and imaged in real-time with fluorescence microscopy. Following a delay of up to 120 seconds, adhesion to PC or APC generated a rapid and sustained Ca²⁺spike, which subsequently declined over a period of 3-10 minutes (FIG. 5). Minimal oscillations were observed during the sustained elevation of Ca²⁺. A distinct pattern of intracellular Ca²⁺was observed in platelets on thrombin. An initial, rapid elevation in intracellular Ca²⁺was followed by a declining phase of Ca²⁺levels that was superimposed by a series of small Ca²⁺oscillations (FIG. 5). In contrast, a series of rhythmic spikes of Ca²⁺, each lasting 4-8 seconds, was observed in platelets on fibrinogen (FG), while the presence of the intracellular Ca²⁺ chelator, BAPTA-AM, abrogated intracellular Ca²⁺elevations in platelets on all surfaces (FIG. 5).

### Example 3

### Molecular mechanisms regulating platelet adhesion and activation

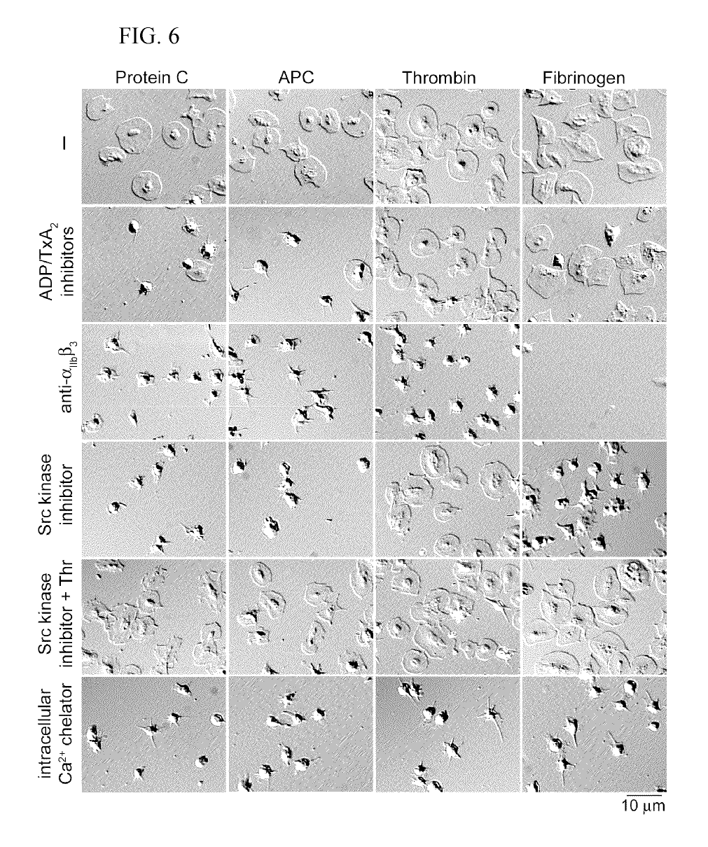

Purified human platelets were exposed to immobilized ligands for 45 minutes at 37° C. in the presence of vehicle, function-blocking antibodies or pharmacological pathway inhibitors. Adherent platelets were fixed and imaged via DIC microscopy. The results showed that addition of the ADP-scavenger apyrase and TxA₂ inhibitor indomethacin resulted in a >60% reduction in platelet adhesion on PC and on APC, as well as a substantial reduction in the degree of platelet lamellipodia formation (FIG. 6 and Table 1). In contrast, apyrase and indomethacin had no effect on platelet adhesion and spreading on thrombin. Moreover, platelet lamellipodia formation on PC, APC or thrombin was abrogated in the presence of the α_{IIb}β₃ mAb eptifibatide (FIG. 6). The presence of either a blocking α_{IIb}β₃ or GPIb mAb, whether alone or in combination, did not affect the degree of platelet adhesion to PC, APC, and thrombin under static conditions. Platelets treated with the Src kinase inhibitor, PP2, failed to form full lamellipodia on PC, APC, and fibrinogen, while the platelets retained full lamellipodia formation on thrombin. The inhibitory action of PP2 was overcome by the exogenous addition of thrombin as evidenced by the ability of platelets to form lamellipodia on PC, APC and fibrinogen in the presence of thrombin in solution subsequent to PP2 treatment. Platelets treated with the intracellular Ca²⁺chelator, BAPTA-AM, were able to form filopodia, but lacked the ability to form lamellipodia on all surfaces. Platelet adhesion to thrombin, but not to PC, APC, or fibrinogen was abrogated in the presence of PPACK (a thrombin inhibitor), which irreversibly inactivates the active site of enzymes. Taken together, the data suggests that PC is capable of supporting pro-hemostatic platelet adhesion and activation in an ADP-dependent manner

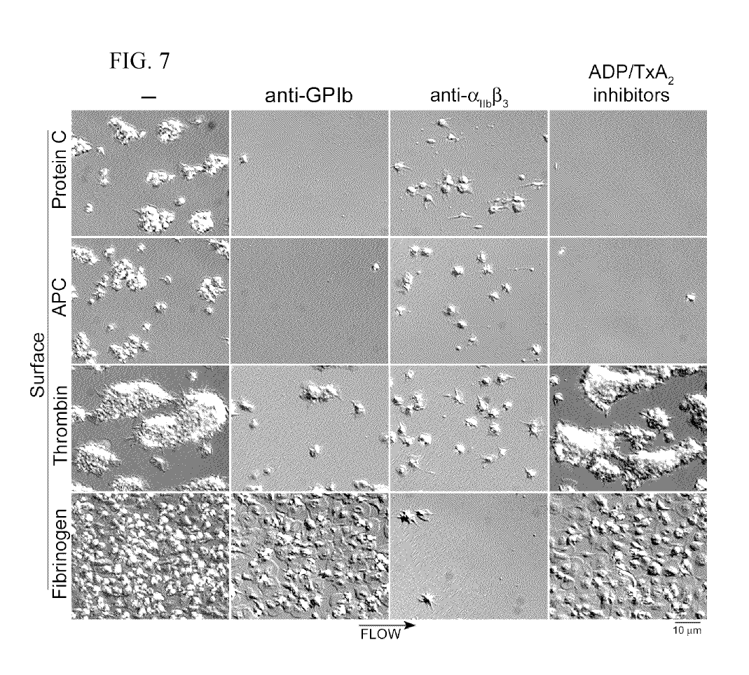

In the majority of previous studies (Rand et al. Transfus. Apher. Sci. 28:307-317, 2003), platelet activation and spreading have been evaluated under static conditions or in the low-shear conditions present in an aggregometer, whereas thrombus formation in vivo occurs under progressively increasing shear due to blood flow through the narrowing lumen. Therefore, to characterize the molecular mechanisms of APC- and PC-platelet interactions under physiologically relevant conditions, a parallel-plate flow chamber was utilized to mimic the shear conditions prevalent in the vasculature. As shown in FIG. 7, immobilized PC or APC supported platelet adhesion and limited the formation of platelet aggregates following a perfusion of reconstituted blood at 300 s⁻¹ for 3 minutes. Moreover, immobilized thrombin supported substantial platelet adhesion and robust aggregation under flow, while immobilized fibrinogen supported a confluent layer of adherent platelet, with only limited platelet aggregation. Platelet recruitment to immobilized PC, APC, and thrombin, but not to fibrinogen, was substantially inhibited in the presence of the function-blocking GPIb mAb 6D1 (10 μg/ml). The formation of platelet aggregates, but not platelet-APC or platelet-PC binding, was eliminated in the presence of an anti-α_{IIb}β₃ mAb. Platelet adhesion on fibrinogen was abrogated in the presence of the anti-α_{IIb}β₃ mAb eptifibatide. The presence of inhibitors to ADP/TxA₂ abrogated platelet deposition on APC and PC, but not on thrombin or fibrinogen. Platelets in sodium-citrate anti-coagulated whole blood failed to adhere to immobilized PC or APC under flow, in contrast to the substantial degree of platelets observed to adhere to thrombin and fibrinogen in whole blood. Platelet adhesion and aggregation was absent on PC, APC, and thrombin, but not fibrinogen, surfaces using PPACK-anticoagulated whole blood (Table 2).

TABLE 2
Platelet adhesion and aggregation under flow*
(expressed as percent surface coverage)

| | Reconstituted | | |
|---|---|---|---|
| Surface | blood | NaCit WB | PPACK WB |
| PC | 10.7 ± 1.2 | 0.2 ± 0.1 | 0.3 ± 0.1 |
| APC | 9.53 ± 1.3 | 0.1 ± 0.1 | 0.2 ± 0.1 |
| thrombin | 54.5 ± 4.1 | 5.7 ± 2.3 | 0.1 ± 0.1 |
| fibrinogen | 88.2 ± 6.1 | 89.1 ± 3.8 | 91.3 ± 2.9 |

*Reconstituted blood or whole blood (WB) anticoagulated with either sodium citrate (NaCit) (0.38%) or PPACK (40 μM) was perfused over surfaces of immobilized PC, APC, thrombin and fibrinogen. Platelet adhesion is expressed as the percentage of surface coverage by platelets. Values are reported as mean ± SEM; n = 3.

Real-time video microscopy studies revealed that PC, APC, and thrombin supported a substantial degree of platelet tethering and rolling under shear conditions using PPACK-anticoagulated whole blood. Human whole blood anticoagulated with PPACK (40 μM) was flowed over surfaces of immobilized PC, APC and thrombin. The mean number of platelets tethering to each surface during 50 sec±SEM is shown in Table 3. Anti-GPIb mAb 6D1 (20 μg/mL) was added to the blood for select experiments. The number of interacting platelets (cells that bound to the surface for >100 msec before rolling along the surface or resuming the velocity of free-flowing non-interacting cells) was significantly reduced in the presence of the anti-GPIb mAb 6D1. These results indicate that GPIb plays a crucial role in mediating platelet binding to APC/PC under shear flow conditions. The results also demonstrated a critical role for the secondary mediators ADP and TxA₂ in mediating platelet adhesion and aggregation to APC/PC under shear conditions.

TABLE 3
Interactions between APC/PC and platelets under flow

| | Suspension | Platelet interaction |
|---|---|---|
| Surface | treatment | (cells/mm²/sec) |
| BSA | — | 32.3 ± 14.6 |
| PC | — | 109.3 ± 21.5 |
| PC | 6D1 | 28.5 ± 7.2* |
| APC | — | 155.5 ± 45.5 |
| APC | 6D1 | 33.7 ± 6.4* |
| thrombin | — | 226.2 ± 145.4 |
| thrombin | 6D1 | 58.5 ± 19.2* |

*p < 0.01 with respect to untreated samples.

### Example 4

### Role of Protein C in Hemostasis

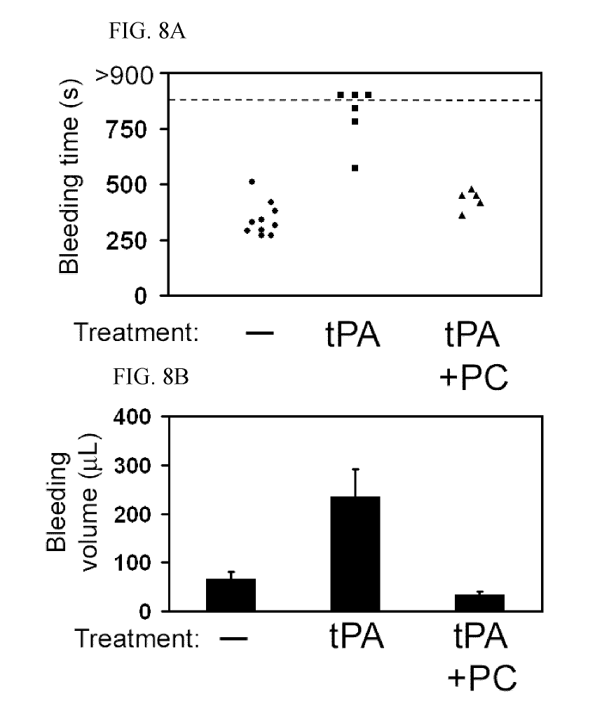

To define the role of PC in hemostasis, tail bleeding assays were performed on mice. Following anesthetization, a 3-mm segment of the tail tip was cut off with a scalpel, and the tail was placed in a microcentrifuge tube of room temperature water. Both the time to cessation of bleeding and the total blood loss were recorded. The experiment was stopped after 900 seconds when the bleeding did not cease naturally. To enhance bleeding, in selected experiments prior to tail clipping, anesthetized mice were infused with tissue-type plasminogen activator (tPA; 2 mg/kg), which activates plasminogen, leading to fibrin degradation and increased bleeding. The results indicated that intravenous injection of murine PC (3 mg/kg) into tPA-treated wild-type mice significantly reduced the bleeding time (FIG. 8A). This reduction was further corroborated by a significant decrease in the amount of blood loss in PC-treated mice (235±56.7 μl compared with 34.4±5.55 μl for tPA-treated mice in the presence of vehicle or PC, respectively) (FIG. 8B). These results suggest that PC plays a pro-hemostatic role in the mouse model of tail bleeding.

The results described herein demonstrate that PC-platelet interactions play a pro-hemostatic role by contributing to platelet activation in a GPIb dependent manner

### Example 5

### Biophysical Characterization of Receptor-Ligand Interactions

The interaction of PC and APC with GPIb was evaluated using surface plasmon resonance (SPR). PC, APC, thrombin and the AI domain of VWF were each perfused over a glycocalicin (the proteolytic fragment of GPIb) coated sensor tip. The sensorgrams at different concentrations were obtained and normalized by subtracting background signals from the glycocalicin surface. Specific and saturable binding of all four ligands to glycocalicin was observed at a range of concentrations from 78 nM to 5.0 μl M. After fitting SPR data to a Langmuir one-to-one binding model, the K_{d} for the interaction of PC and APC with glycocalicin was calculated at 330 and 308 nM, respectively, which is nearly 10-fold less than that between GPIb and VWF A1 (Table 4). Taken together, these findings demonstrate that GPIb serves as a receptor for PC and APC.

TABLE 4
Dissociation constants for glycocalicin

| Ligand | K_{d} |
|---|---|
| PC | 330 ± 26 nM |
| APC | 308 ± 21 nM |
| thrombin | 361 ± 29 nM |
| VWF A1 | 36 ± 4.0 nM |

To characterize APC- and PC-platelet interactions, the biophysical parameters associated with APC/PC binding to the platelet surface was determined by immobilizing PC onto a 0.99-μm carboxylate-modified latex bead (PC-bead). An individual platelet was then trapped from a suspension of washed platelets (5×10⁶ plt/ml) and PC-beads (2×10⁵/ml), and was manually attached to a 5-μm diameter fibrinogen-coated silica bead that had been immobilized onto a glass surface. Subsequently, a PC- bead, trapped by the laser light, was brought within a distance of 2-3 μm from the immobilized platelet. Oscillation of the PC-bead was initiated at 50 Hz with a 0.8 μm peak-to-peak amplitude, and then the bead was brought into contact with the platelet by micromanipulation of the stage. The lateral forces that the trap exerted on the bead were measured with a quadrant detector conjugated to the back focal plane of the condenser and were calibrated from the low-frequency component of the Brownian motion.

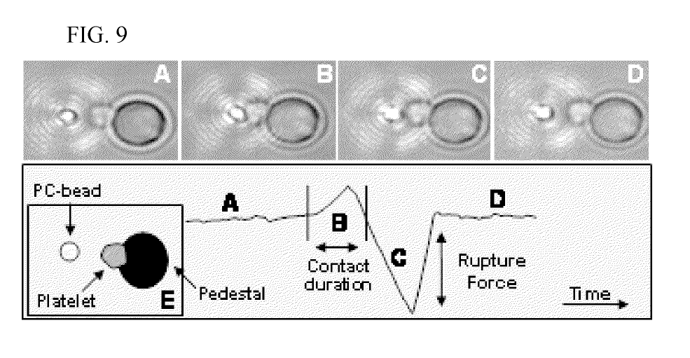

FIG. 9 shows a representative data trace for a typical interaction between a PC-bead and an immobilized platelet, partitioned into four parts. The PC-bead, trapped near the center of the laser beam, is moved toward (Upper A) or away (Upper D) from the immobilized platelet, corresponding to zero force (Lower A,D). At the moment of contact (Upper B), the platelet stops the motion of the PC-bead while the laser beam continues in the same direction (right). The laser trap exerts a positive, compressive force on the platelet and bead (Lower B). The trap motion is then reversed, and the compressive force declines to zero. Peak B (Lower) represents the time that the platelet and the PC-bead are under a compressive force (contact duration time). When the PC-bead and platelet unite (Upper C), the bead position remains nearly constant as the laser continues to move to the left. The force on the bead increases in the negative direction (Lower C), almost linearly until the PC-bead-platelet bond is ruptured and the force rapidly returns to nearly zero. These results suggest that PC is capable of binding platelets under non-equilibrium conditions.

### Example 6

### Characterization of PC Binding to the Platelet Surface

To characterize the ability of platelets to recruit PC to the platelet surface and to catalyze the APC generation, platelets were immobilized onto a glass slide treated with 3-aminopropyltriethoxysilane (APES). The platelet-coated slides were treated with 1% BSA for 10 min prior to their use in adhesion assays to prevent non-specific interactions. PC was immobilized onto the surface of 10 μm-diameter polystyrene beads (PC-beads). Using a parallel-plate flow chamber, PC-beads were perfused over immobilized platelets at a shear rate of 150/second for 5 minutes.

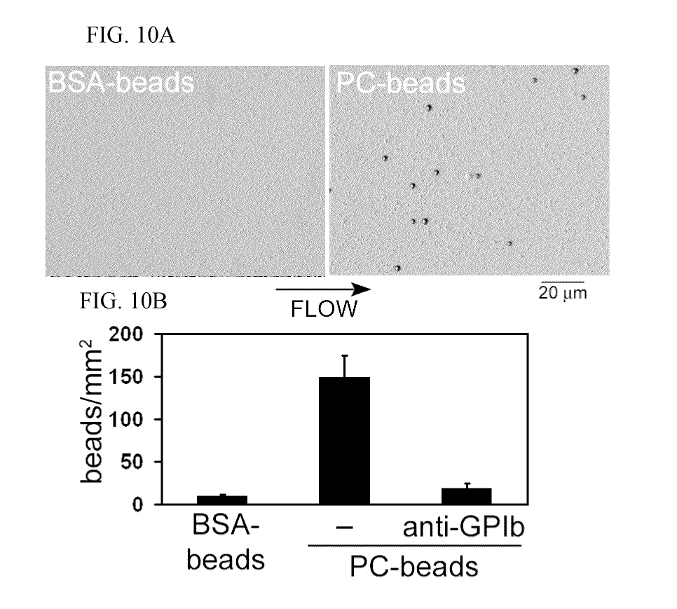

As shown in FIG. 10, immobilized platelets supported the recruitment and firm adhesion of PC-coated beads. The presence of a blocking GPIb mAb 6D1 prevented PC-bead attachment to platelets under shear flow conditions. Beads coated with BSA failed to bind to immobilized platelets. These results indicate that platelets support a GPIb-dependent recruitment of PC to the platelet surface under shear.

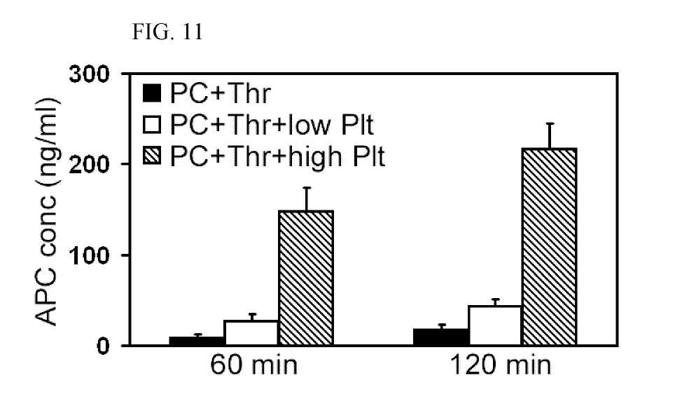

It is believed that, in addition to promoting pro-hemostatic platelet activation at sites of injury, platelets facilitate the anti-thrombotic local generation of APC. To characterize the ability of platelet receptors to potentiate the conversion of PC to APC, a suspension of 50 μl of platelets (at both 7×10⁸/ml (low platelet count) and 4.5×10⁹/ml (high platelet count)) was incubated with 100 nM PC in the presence of thrombin. Reactions were stopped after 60-120 minutes though the addition of 100 nM hirudin, and levels of APC were determined using an APC-specific mAb in conjunction with a HAPC-1555 enzyme capture assay (Liaw et al. J. Thromb. Haemost. 1:662-670, 2003). These data demonstrate that the presence of activated platelets potentiated a 3-fold and 15-fold increase in the concentration of (unbound) APC in the fluid phase at low and high platelet concentrations, respectively (FIG. 11). This assay indicates that the thrombin-catalyzed activation of PC is augmented by the platelet surface.

Taken altogether, these data indicate that APC- and PC-platelet interactions play a dual role during hemostasis and thrombosis, and provide evidence that PC-platelet binding is pro-hemostatic, while platelet facilitated APC generation is anti-thrombotic.

### Example 7

### Effect of Wild-Type PC and an Active Site PC Mutant on Hemostasis

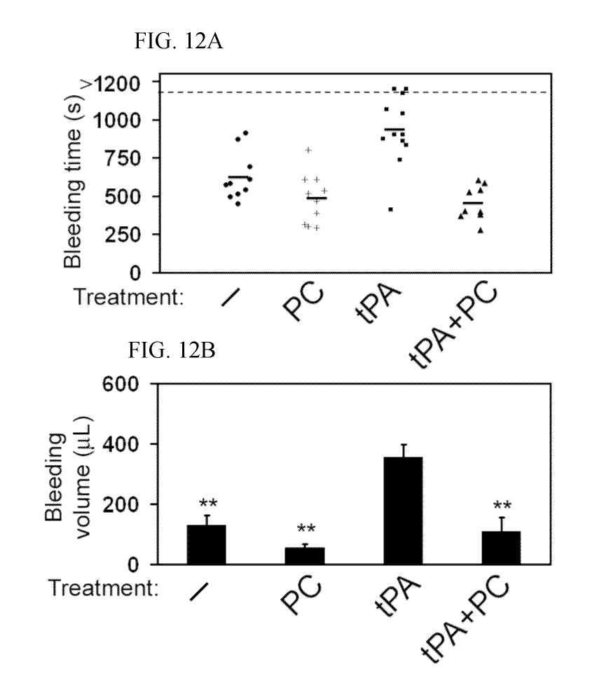

This example further describes the hemostatic activity of PC and demonstrates that a variant of PC retains hemostatic activity. Murine tail bleeding assays were used to assess the effect of elevated levels of plasma protein C on hemostasis. The effects of tPA (2 mg/kg) and/or recombinant murine protein C (3 mg/kg) on tail bleeding assays are summarized in FIG. 12. The baseline mean bleeding time (623±49.3 seconds; n=10) was significantly prolonged following administration of the fibrinolytic agent tPA (932±65.3 seconds; n=12, p<0.05). Bleeding volume following tPA administration (357±41.2 μL; n=12) was also increased compared to vehicle control (130±32.1 μL; n=10; p<0.01). Infusion of murine protein C with vehicle reduced bleeding time to 483±51.7 seconds and blood loss to 56.8±11.4 .μL (n=10; p<0.01). Co-administration of protein C with tPA reduced the bleeding time (454±35.6 seconds; n=10; p<0.01) and bleeding volume (109±45.6 μL; n=10; p<0.01) to near vehicle control levels.

A similar reduction in bleeding time and volume was observed following co-infusion of tPA with the recombinant murine form of the enzymatically inactive (active site mutant S360A) protein C (578±35.6 seconds and 81.8±36.4 μL for bleeding time and volume, respectively; n=8; p<0.01). These data suggest that recombinant protein C acts as a hemostatic agent during tPA-induced hemostasis impairment in mice, and that the enzymatic active site of protein C is not required for this effect.

### Example 8

### Treatment of a Bleeding Disorder with a Protein C Polypeptide

This example describes the treatment of a patient diagnosed with hemophilia with a human recombinant protein C polypeptide. A patient diagnosed with hemophilia is treated prophylactically with human protein C polypeptide by administration of purified protein C polypeptide having an amino acid sequence of SEQ ID NO: 2 in a pharmaceutically acceptable carrier. Protein C is administered to the patient intravenously by bolus injection at a dose of 3 mg/kg once a week. The dose and dosing schedule of protein C administration can vary and is determined in part by the severity of the disease, and the age, weight and general health of the patient. Patients having moderate to severe hemophilia prone to episodes of spontaneous bleeding, can receive repeated doses at regular intervals, such as once a day, twice a week, once a week, twice a month, or once a month. An appropriate dose and timing of administration can be determined by a skilled practitioner.

A patient with mild hemophilia that exhibits uncontrolled bleeding in response to trauma or surgery can be treated with protein C as needed. For example, a patient diagnosed with mild hemophilia that requires surgery can be treated with protein C prior to surgery. The patient with mild hemophilia is administered purified recombinant human protein C having an amino acid sequence of SEQ ID NO: 2 in a pharmaceutically acceptable carrier. Protein C is administered by intravenous bolus injection at a dose of 3 mg/kg approximately one hour prior to surgery, and every twelve hours following surgery as needed. The patient can be monitored for uncontrolled bleeding and treated with additional protein C as needed.

### Example 9

### Treatment of a Bleeding Episode with a Protein C Polypeptide

The example describes the treatment of a patient having a traumatic wound with a human recombinant protein C polypeptide. A patient presenting with a stab wound is first treated surgically to close the wound. The patient is then treated with human protein C polypeptide by administration of purified protein C polypeptide having an amino acid sequence of SEQ ID NO: 2 in a pharmaceutically acceptable carrier. Protein C is administered to the patient intravenously by bolus injection at a dose of 3 mg/kg every 12 hours as needed. The dose and dosing schedule of protein C administration can vary and is determined in part by the severity of the wound, and the age, weight and general health of the patient. An appropriate dose and administration schedule can be determined by a skilled practitioner. For example, the patient can be administered protein C every 12 hours until bleeding is controlled.

This disclosure provides methods of treating a bleeding disorder or bleeding episode by administration of a mammalian protein C polypeptide or polynucleotide. The disclosure further provides protein C polypeptides and polynucleotides for use as hemostatic agents. It will be apparent that the precise details of the methods described may be varied or modified without departing from the spirit of the described disclosure. We claim all such modifications and variations that fall within the scope and spirit of the claims below.

1. A method of promoting hemostasis in a subject, comprising administering to the subject a protein C polypeptide, or a hemostatic fragment or variant thereof, in a therapeutically effective dose sufficient to achieve hemostasis.

2. Use of a protein C polypeptide, or a hemostatic fragment or variant thereof in a method of promoting hemostasis in a subject, wherein the method comprises administering to the subject the protein C polypeptide, or hemostatic fragment or variant thereof, in a therapeutically effective dose sufficient to achieve hemostasis.

3. The method of claim 1 or claim 2, wherein the subject has been diagnosed with a bleeding disorder or a bleeding episode, and wherein administration of the protein C polypeptide or hemostatic fragment or variant thereof therapeutically improves the bleeding disorder or the bleeding episode.

4. The method of any one of claims 1-3, wherein the protein C polypeptide or hemostatic fragment or variant thereof comprises at least 90% sequence identity with the amino acid sequence set forth as SEQ ID NO: 2, 4, 6, 8, 10, 12, 14, 16 or 18.

5. The method of any one of claims 1-3, wherein the protein C polypeptide or hemostatic fragment or variant thereof comprises at least 95% sequence identity with the amino acid sequence set forth as SEQ ID NO: 2, 4, 6, 8, 10, 12, 14, 16 or 18.

6. The method of any one of claims 1-3, wherein the protein C polypeptide or hemostatic fragment or variant thereof comprises at least 99% sequence identity with the amino acid sequence set forth as SEQ ID NO: 2, 4, 6, 8, 10, 12, 14, 16 or 18.

7. The method of any one of claims 1-3, wherein the protein C polypeptide comprises the amino acid sequence set forth as SEQ ID NO: 2, 4, 6, 8, 10, 12, 14, 16 or 18.

8. The method of any one of claims 1-3, wherein the protein C polypeptide consists of the amino acid sequence set forth as SEQ ID NO: 2, 4, 6, 8, 10, 12, 14, 16 or 18.

9. The method of any one of claims 1-3, wherein the protein C polypeptide variant is the S360A mutant.

10. The method of any one of claims 1-9, wherein the protein C polypeptide or hemostatic fragment or variant thereof is administered by a parenteral route.

11. The method of any one of claims 1-10, wherein the protein C polypeptide or hemostatic fragment or variant thereof is administered intravenously.

12. The method of any one of claims 1-9, wherein the protein C polypeptide or hemostatic fragment or variant thereof is administered topically.

13. The method of claim 12, wherein the protein C polypeptide or hemostatic fragment or variant thereof is administered as part of a wound dressing.

14. The method of any one of claims 1-13, wherein the protein C polypeptide or hemostatic fragment or variant thereof is administered at a dose of about 1 to about 100 mg/day.

15. The method of any one of claims 1-13, wherein the protein C polypeptide or hemostatic fragment or variant thereof is administered at a dose of about 1 to about 10 mg/kg.

16. The method of any one of claims 1-15, wherein the protein C polypeptide or hemostatic fragment or variant thereof is administered in a single dose.

17. The method of any one of claims 1-15, wherein the protein C polypeptide or hemostatic fragment or variant thereof is administered in multiple doses.

18. The method of any one of claims 1-3, wherein administering the protein C polypeptide comprises administering a vector comprising a protein C nucleic acid sequence, wherein the protein C nucleic acid sequence encodes a protein C polypeptide or a hemostatic fragment or variant thereof.

19. The method of claim 18, wherein the nucleic acid sequence comprises at least 90% sequence identity with the nucleotide sequence set forth as SEQ ID NO: 1, 3, 5, 7, 9, 11, 13, 15 or 17.

20. The method of claim 18, wherein the nucleic acid sequence comprises at least 95% sequence identity with the nucleotide sequence set forth as SEQ ID NO: 1, 3, 5, 7, 9, 11, 13, 15 or 17.

21. The method of claim 18, wherein the nucleic acid sequence comprises at least 99% sequence identity with the nucleotide sequence set forth as SEQ ID NO: 1, 3, 5, 7, 9, 11, 13, 15 or 17.

22. The method of claim 18, wherein the nucleic acid sequence comprises SEQ ID NO: 1, 3, 5, 7, 9, 11, 13, 15 or 17.

23. The method of claim 18, wherein the nucleic acid sequence consists of SEQ ID NO1, 3, 5, 7, 9, 11, 13, 15 or 17.

24. The method of claim 18, wherein the protein C polypeptide variant is the S360A mutant.

25. The method of any one of claims 18-24, wherein the vector is a viral vector.

26. The method of any one of claims 18-24, wherein the vector is a eukaryotic expression vector.

27. The method of any one of claims 18-26, wherein the vector is administered by a parenteral route.

28. The method of any one of claims 18-27, wherein the vector is administered intravenously.

29. The method of any one of claims 18-28, wherein the vector is administered in a single dose.

30. The method of any one of claims 18-28, wherein the vector is administered in multiple doses.

31. The method of any one of claims 3-30, wherein the bleeding disorder is a clotting factor deficiency, a platelet disorder, thrombocytopenia, vitamin K deficiency or von Willebrand's disease.

32. The method of claim 31, wherein the clotting factor deficiency is hemophilia A, hemophilia B or hemophilia C.

33. The method of any one of claims 3-30, wherein the bleeding episode is caused by a drug, an anticoagulant overdose, an aneurysm, blood vessel rupture, surgery, a traumatic injury, cancer, gastrointestinal ulceration or an infection.


SEQUENCE LISTING (18 sequences)

SEQ ID NO: 1 - DNA, 1776 bp, Homo sapiens
  CDS (74)..(1459)
gctgtcatggcggcaggacggcgaacttgcagtatctccacgacccgcccctacaggtgccagtgcctccagaatgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttagcgaccctccctgcagggctgggcttttgcatggcaatggatgggacattaaagggacatgtaacaagcacaccggcctgctgttctgtccttccatccctcttttgggctcttctggagggaagtaacatttactgagcacctgttgtatgtcacatgccttatgaatagaatcttaactcctagagcaactctgtggggtggggaggagcagatccaagttttgcggggtctaaagctgtgtgtgttgagggggatactctgtttatgaaaaagaataaaaaacacaaccacgaaaaaaaaaaaaaaaaaaaaaaa

SEQ ID NO: 2 - protein, 461 aa, Homo sapiens
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 3 - DNA, 1792 bp, Homo sapiens
  CDS (56)..(1441)
cggcgaacttgcagtatctccacgacccgcccctacaggtgccagtgcctccagaatgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctctctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttagcgaccctccctgcagggctgggcttttgcatggcaatggatgggacattaaagggacatgtaacaagcacaccggcctgctgttctgtccttccatccctcttttgggctcttctggagggaagtaacatttactgagcacctgttgtatgtcacatgccttatgaatagaatcttaactcctagagcaactctgtggggtggggaggagcagatccaagttttgcggggtctaaagctgtgtgtgttgagggggatactctgtttatgaaaaagaataaaaaacacaaccacgaaaaaaaaaaaaaaaaaaaaaaaaaaaaaaaaaaaaaaaaaaaaaaaaaaaaaaaaa

SEQ ID NO: 4 - protein, 461 aa, Homo sapiens; identical to SEQ ID NO: 2

SEQ ID NO: 5 - DNA, 1366 bp, Homo sapiens
ccaagggcacggcacgtgcatcgacggcatcggcagcttcagctgcgactgccgcagcggctgggagggccgcttctgccagcgcgaggtgagcttcctcaattgctctctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggacgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttagcgaccctccctgcagggctgggcttttgcatggcaatggatgggacattaaagggacatgtaacaagcacaccggcctgctgttctgtccttccatccctcttttgggctcttctggagggaagtaacatttactgagcacctgttgtatgtcacatgccttatgaatagaatcttaactcctagagcaactctgtcgggtggggaggagcagatccaagttttgcggggtctaaagctgtgtgtgttgagggggatactctgtttatgaaaaagaataaaaaacacaaccacg

SEQ ID NO: 6 - protein, 356 aa, Homo sapiens
QGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 7 - DNA, 1499 bp, Mus musculus
  CDS (11)..(1396)
gagctccaggatgtggcaattcagagtcttcctgctgctcatgtccacctggggaatatctagcataccggcccatcctgacccagtgttctccagcagcgagcatgcccaccaggtgcttcgggtcagacgtgccaacagcttcctggaagagatgcggccaggcagcctggaacgggagtgtatggaggagatctgtgacttcgaggaggcccaggagattttccaaaatgtggaagacacactggccttctggatcaagtactttgacggtgaccagtgctcggctccacccttggaccaccagtgcgacagcccatgctgcgggcatggcacttgcatcgacggcataggcagcttcagctgcagctgcgataagggctgggagggcaagttctgtcagcaggagttgcgcttccaggactgtcgggtgaacaatggcggctgcttgcactactgcctggaggagagcaatgggcggcgctgcgcttgtgccccgggctatgagctggcagacgaccacatgcgctgcaagtccactgtgaattttccatgtgggaaactggggaggtggatagagaagaaacgcaagatcctcaaacgagacacagacttagaagatgaactggaaccagatccaaggatagtcaacggaacgctgacgaagcagggtgacagtccttggcaggcaatccttctggactccaagaagaagctggcctgcggaggggtgctcatccacacttcctgggtgctgacggcagcccactgcgtggagggcaccaagaagcttaccgtgaggcttggtgagtatgatctgcgacgcagggaccactgggagctggacctggacatcaaggagatcctcgtccaccctaactacacccggagcagcagtgacaacgacattgctctgctccgcctagcccagccagccactctctccaaaaccatagtgcccatctgcctgccgaacaatgggctcgctcagcaggagctcactcaggctggccaggagacagtggtgacaggctggggctatcaaagcgacagaatcaaggatggcagaaggaaccgcaccttcatcctcaccttcatccgcatccctttggttgctcgaaatgagtgcgtggaggtcatgaagaatgtggtctcggagaacatgctgtgtgcaggcatcattgggaacacgagagacgcctgtgatggtgacagtggggggcccatggtggtcttctttcggggtacctggttcctggtgggcctggtgagctggggtgagggctgtgggcacaccaacaactatggcatctacaccaaagtgggaagctacctcaaatggattcacagttacattggggaaaagggtgtctcccttaagagccagaagctatagcacccctccctgctcacctctggaccctagaagtcactcttggagtaaggctgggctagtgagtaccaagacagaggacattaaaggagcatgcaacaaacat

SEQ ID NO: 8 - protein, 461 aa, Mus musculus
MWQFRVFLLLMSTWGISSIPAHPDPVFSSSEHAHQVLRVRRANSFLEEMRPGSLERECMEEICDFEEAQEIFQNVEDTLAFWIKYFDGDQCSAPPLDHQCDSPCCGHGTCIDGIGSFSCSCDKGWEGKFCQQELRFQDCRVNNGGCLHYCLEESNGRRCACAPGYELADDHMRCKSTVNFPCGKLGRWIEKKRKILKRDTDLEDELEPDPRIVNGTLTKQGDSPWQAILLDSKKKLACGGVLIHTSWVLTAAHCVEGTKKLTVRLGEYDLRRRDHWELDLDIKEILVHPNYTRSSSDNDIALLRLAQPATLSKTIVPICLPNNGLAQQELTQAGQETVVTGWGYQSDRIKDGRRNRTFILTFIRIPLVARNECVEVMKNVVSENMLCAGIIGNTRDACDGDSGGPMVVFFRGTWFLVGLVSWGEGCGHTNNYGIYTKVGSYLKWIHSYIGEKGVSLKSQKL

SEQ ID NO: 9 - DNA, 1661 bp, Mus musculus
  CDS (163)..(1545)
ggttatggattaacctgactcccagactgacatggcgctacctggacgaaattgcagtttctccttggcccacgcctgtgtaggcttgtgacaagccgcgtatctcctccaagcctttgggtcccttcccatgaaatggagacaggtgtcagcagctccaggatgtggcaattcagagtcttcctgctgctcatgtccacctggggaatatctagcataccggcccatcctgacccagtgttctccagcagcgagcatgcccaccaggtgcttcgggtcagacgtgccaacagcttcctggaagagatgcggccaggcagcctggaacgggagtgtatggaggagatctgtgacttcgaggaggcccaggagattttccaaaatgtggaagacacactggccttctggatcaagtactttgacggtgaccagtgctcggctccacccttggaccaccagtgcgacagcccatgctgcgggcatggcacttgcatcgacggcataggcagcttcagctgcagctgcgataagggctgggagggcaagttctgtcagcaggagttgcgcttccaggactgtcgggtgaacaatggcggctgcttgcactactgcctggaggagagcaatgggcggcgctgcgcttgtgccccgggctatgagctggcagacgaccacatgcgctgcaagtccactgtgaattttccatgtgggaaactggggaggtggatagagaagaaacgcaagatcctcaaacgagacacagacttagaagatgaactggaaccagatccaaggatagtcaacggaacgctgacgaagcagggtgacagtccttggcaggcaatccttctggactccaagaagaagctggcctgcggaggggtgctcatccacacttcctgggtgctgacggcagcccactgcgtggagggcaccaagaagcttaccgtgaggcttggtgagtatgatctgcgacgcagggaccactgggagctggacctggacatcaaggagatcctcgtccaccctaactacacccggagcagcagtgacaacgacattgctctgctccgcctagcccagccagccactctctccaaaaccatagtgcccatctgcctgccgaacaatgggctggcgcaggagctcactcaggctggccaggagacagtggtgacaggctggggctatcaaagcgacagaatcaaggatggcagaaggaaccgcaccttcatcctcaccttcatccgcatccctttggttgctcgaaatgagtgcgtggaggtcatgaagaatgtggtctcggagaacatgctgtgtgcaggcatcattggggacacgagagacgcctgtgatggtgacagtggggggcccatggtggtcttctttcggggtacctggttcctggtgggcctggtgagctggggtgagggctgtgggcacaccaacaactatggcatctacaccaaagtgggaagctacctcaaatggattcacagttacattggggaaaagggtgtctcccttaagagccagaagctatagcacccctccctgctcacctctggaccctagaagtcactcttggagtaaggctgggctagtgagtaccaagacagaggacattaaaggagcatgcaacaaacataaaaaaaaaaaaa

SEQ ID NO: 10 - protein, 460 aa, Mus musculus
MWQFRVFLLLMSTWGISSIPAHPDPVFSSSEHAHQVLRVRRANSFLEEMRPGSLERECMEEICDFEEAQEIFQNVEDTLAFWIKYFDGDQCSAPPLDHQCDSPCCGHGTCIDGIGSFSCSCDKGWEGKFCQQELRFQDCRVNNGGCLHYCLEESNGRRCACAPGYELADDHMRCKSTVNFPCGKLGRWIEKKRKILKRDTDLEDELEPDPRIVNGTLTKQGDSPWQAILLDSKKKLACGGVLIHTSWVLTAAHCVEGTKKLTVRLGEYDLRRRDHWELDLDIKEILVHPNYTRSSSDNDIALLRLAQPATLSKTIVPICLPNNGLAQELTQAGQETVVTGWGYQSDRIKDGRRNRTFILTFIRIPLVARNECVEVMKNVVSENMLCAGIIGDTRDACDGDSGGPMVVFFRGTWFLVGLVSWGEGCGHTNNYGIYTKVGSYLKWIHSYIGEKGVSLKSQKL

SEQ ID NO: 11 - DNA, 1543 bp, Rattus norvegicus
  CDS (49)..(1434)
cgaaattgcagtttctccttggcccacccctgtgtcagcagctccaggatgtggcagttcagaatcttcctgctgttcgcgtccacctgggggatttctggcgtatcagcccatcccgacccagtgttctccagcagcgagggtgcccaccaggtgcttcgggtcagacgagccaacagcttcctggaggaggtgcgggcaggcagcctggagcgggagtgtatggaggagatctgtgacttcgaggaggcccaggagattttccagaatgtggaagacacactggccttttggatcaagtacttcgatggtgaccagtgctcaactccgcccttggaccaccaatgcgacagcccatgctgcggccatggcacatgcatcgacggcctgggcggcttcagctgcagctgcgataagggctgggagggcaggttctgtcagcaggagatgggcttccaggactgtcgggtgaaaaatggcggctgctaccactactgcctggaggagaccagagggcggcgctgccgttgcgccccgggctatgagctggcagatgaccacatgcactgcaggcccaccgtgaattttccgtgtgggaaactgtggaagcggactgacaagaaacgcaagaacttcaaacgggacatagacccagaagacgaagaactagaactaggtccaaggatagtcaatggaacactaacaaagcagggtgacagtccctggcaggcgatccttctggactccaagaagaagctagcctgtggaggggtgctcatccacacctcctgggtgctgacggcagcccactgtctggagagcagcaagaagcttaccgtgaggcttggtgagtatgatctgagacgcagggacccctgggagttggacctggacatcaaggaggtcctcgtccaccctaactacacccggagcaacagcgacaacgacatcgccctgctccgcctgtcccagccagccacactctctaaaaccatagtgcccatctgtctgccgaacagcggcctggcgcaggagctcagtcaggctggccaggagacggtggtgacaggctggggctatcaaagcgacaaagtcaaggatggcagaaggaaccgcacctttattctcaccttcatccgcatccctttggccgctcgaaatgactgcatgcaggtcatgaacaacgtggtctcggagaacatgctctgcgccggcatcattggagacacgagagacgcctgcgacggcgacagtgggggacctatggtggtcttctttcggggtacctggtttctggtgggcctggtgagctggggtgagggctgtgggcacctcaacaactatggcgtctacaccaaagtgggtagctacctcaaatggatccacagctacataggggaaagggatgtttccctgaagagcccgaagctgtagcatccctccctgctcatctctggggcccagaggtcactcttagaataaggctgggctagtgagtaccaagacaggggacattaaaggggcaagcaacacctgaaaaaaa

SEQ ID NO: 12 - protein, 461 aa, Rattus norvegicus
MWQFRIFLLFASTWGISGVSAHPDPVFSSSEGAHQVLRVRRANSFLEEVRAGSLERECMEEICDFEEAQEIFQNVEDTLAFWIKYFDGDQCSTPPLDHQCDSPCCGHGTCIDGLGGFSCSCDKGWEGRFCQQEMGFQDCRVKNGGCYHYCLEETRGRRCRCAPGYELADDHMHCRPTVNFPCGKLWKRTDKKRKNFKRDIDPEDEELELGPRIVNGTLTKQGDSPWQAILLDSKKKLACGGVLIHTSWVLTAAHCLESSKKLTVRLGEYDLRRRDPWELDLDIKEVLVHPNYTRSNSDNDIALLRLSQPATLSKTIVPICLPNSGLAQELSQAGQETVVTGWGYQSDKVKDGRRNRTFILTFIRIPLAARNDCMQVMNNVVSENMLCAGIIGDTRDACDGDSGGPMVVFFRGTWFLVGLVSWGEGCGHLNNYGVYTKVGSYLKWIHSYIGERDVSLKSPKL

SEQ ID NO: 13 - DNA, 1569 bp, Bos taurus
  CDS (18)..(1460)
tcgagttccaggctgtcatggcggcaggagggcaaccttgcagtttctccgcggcccaccccagtggctgcagcttcagaatgtggcagcttacaagcctcttactgttcgtgaccatctggggaatttccagcacaccagctcctcctgactcagtgttctccagcagccagcgtgcccaccaagtgctgcggatccgcaaacgtgccaactccttcctggaggagctgcggcccggcaacgtggagcgtgagtgctcagaggaggtctgtgagttcgaggaagctcgggagattttccaaaacacggaagacacaatggccttctggtccaagtatagcgacggggaccagtgcgaggaccggccctcagggagcccgtgcgacctcccatgctgtggacgcggcaagtgcatcgatggcctgggcggcttccgctgcgactgcgcggagggctgggagggccgcttctgcttgcacgaggtgcgcttctccaactgctcggcggaaaacggcggctgcgcccactactgcatggaggaggagggccggcgccactgcagctgcgcgcccggctaccggctggaggacgaccaccagctctgcgtgtccaaggtgacgttcccttgtgggaggctagggaagcgaatggagaagaaacgcaagaccttgaaacgtgacacaaaccaagtcgaccaaaaagaccagttggatccacggattgtcgatgggcaggaggctggatggggagagagcccctggcaggcagtgctgctggactccaagaagaagctggtctgtggggcagtgctaatccacgtctcctgggtgctgacggtggcccactgcttggacagccgcaagaagctcatcgtcaggctcggggagtatgacatgcggcgctgggagagctgggaggtggacctggacatcaaggaggtcatcatccaccctaactataccaagagcaccagtgacaacgacatcgccctgctccgcctggccaagcctgccactctttcgcagaccattgtgcccatctgtctcccggatagtggcctctctgagcgcaagctcacccaggtcggccaggagactgtggtgacaggctggggctaccgtgacgagaccaagagaaaccgcaccttcgtcctcagcttcatcaaggtccctgtggtcccgtacaatgcatgtgtccatgccatggaaaacaagatctctgagaacatgctgtgcgctggtatcctcggggacccgagggatgcctgtgagggcgacagtggggggcctatggtcaccttcttccgtggcacctggttcctggtgggcctggtgagctggggcgagggctgtgggcgcctctacaactacggcgtttacaccaaagtcagccgttaccttgactggatctacggccacatcaaagctcaggaggcccctcttgagagccaggtgccttagcatcccccgtcacttgtgtctgggccccagaggacacccttggataggggctggattgttgaatggcaagatggtggacattaaaaaagggcttgctgcaagcacacca

SEQ ID NO: 14 - protein, 480 aa, Bos taurus
MAAGGQPCSFSAAHPSGCSFRMWQLTSLLLFVTIWGISSTPAPPDSVFSSSQRAHQVLRIRKRANSFLEELRPGNVERECSEEVCEFEEAREIFQNTEDTMAFWSKYSDGDQCEDRPSGSPCDLPCCGRGKCIDGLGGFRCDCAEGWEGRFCLHEVRFSNCSAENGGCAHYCMEEEGRRHCSCAPGYRLEDDHQLCVSKVTFPCGRLGKRMEKKRKTLKRDTNQVDQKDQLDPRIVDGQEAGWGESPWQAVLLDSKKKLVCGAVLIHVSWVLTVAHCLDSRKKLIVRLGEYDMRRWESWEVDLDIKEVIIHPNYTKSTSDNDIALLRLAKPATLSQTIVPICLPDSGLSERKLTQVGQETVVTGWGYRDETKRNRTFVLSFIKVPVVPYNACVHAMENKISENMLCAGILGDPRDACEGDSGGPMVTFFRGTWFLVGLVSWGEGCGRLYNYGVYTKVSRYLDWIYGHIKAQEAPLESQVP

SEQ ID NO: 15 - DNA, 1514 bp, Sus scrofa
  CDS (22)..(1401)
cgttgctgtcgcaactccagaatgtggcaacttgcaagcctcttactgctcctgatcatctgggcagtttccagcacaccagttcctcctgactcagtgttctccagcagccagcgggcccaccagatgctgcgcagcaaacgcgccaactccttcctggaggagctgcggcccagcagcctggagcgtgagtgcaaggaggagacctgtgatttcgaggaggctcgggagattttccaaaacacggaaaacacaatggccttctggtccaagtaccatgacggggaccagtgcgcggtctcgccccctgagcacctgtgcgacagcccgtgctgcgggcgtggcacctgtatcgacggcctgggcggcttccgctgcgactgcgctcagggctgggagggccgcttctgcctgcacgaggtgcgcttctccaactgctcgacggaaaacggtggctgtgcgcactactgcctggaggaggagggcgggcgccgctgcgcttgcgcgccaggctaccggctgggggacgaccacctgcagtgcgagccaaaggtgaggtccccttgcgggaggctagggaatcgcatggagaagaaacgcaagaacttgaagcgtgatacagaccaagttgacaaaaaagaagaccaaatagatccgaggctcgtcaatgggaagcagtccccatggggagagagcccctggcaggtgatcctgctggactcaaagaagaagctggcctgtggggcagtgctcatccatgtctcctgggtgctgacagcggcccactgcttggacgactacaagaagctcactgtcaggctcggcgaatatgatctgaggcgtcgggaaaaatgggaggtagacctggacatcaaggagttcctcgtccaccctaactacaccaggagcaccagtgacaatgacatcgccctgctccgcctggccgaaccggccactttctcgcagaccatcgtgcccatctgcctcccagacagtggcctctctgagcgtgagctcacccgggttggccaagagacggtggtgaccggctggggctaccgcagcgaggccaagacaaaccgcagcttcatcctcaacttcatcaaggtccctgtggccccgcacaatgagtgcgtccaggccatgcacaacaagatctctgagaacatgctgtgtgcaggcatcctgggggactctcgtgatgcctgcgagggcgacagcggggggcctatggtggcctccttccgcggcacctggttcctggtgggcctggtgagctggggtgagggctgtgggcgcctccacaactatggcgtttacaccaaagtcagccgttacctcgactggatccatggccacatcagaatggaggaggccttccacaagaaccaggtgccttagtgtctccctaccccactgcttgggccccaggggccatcctcgggtgggggctggtttgttgaatggcaacagtaggacaataaaggggcatgcagtaagcaaaaaaaaaaaaa

SEQ ID NO: 16 - protein, 459 aa, Sus scrofa
MWQLASLLLLLIIWAVSSTPVPPDSVFSSSQRAHQMLRSKRANSFLEELRPSSLERECKEETCDFEEAREIFQNTENTMAFWSKYHDGDQCAVSPPEHLCDSPCCGRGTCIDGLGGFRCDCAQGWEGRFCLHEVRFSNCSTENGGCAHYCLEEEGGRRCACAPGYRLGDDHLQCEPKVRSPCGRLGNRMEKKRKNLKRDTDQVDKKEDQIDPRLVNGKQSPWGESPWQVILLDSKKKLACGAVLIHVSWVLTAAHCLDDYKKLTVRLGEYDLRRREKWEVDLDIKEFLVHPNYTRSTSDNDIALLRLAEPATFSQTIVPICLPDSGLSERELTRVGQETVVTGWGYRSEAKTNRSFILNFIKVPVAPHNECVQAMHNKISENMLCAGILGDSRDACEGDSGGPMVASFRGTWFLVGLVSWGEGCGRLHNYGVYTKVSRYLDWIHGHIRMEEAFHKNQVP

SEQ ID NO: 17 - DNA, 1776 bp, Homo sapiens
  CDS (74)..(1459)
gctgtcatggcggcaggacggcgaacttgcagtatctccacgacccgcccctacaggtgccagtgcctccagaatgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtctgccttggagagtatgacctgcggcgctgggagaagtgggagctgtgcctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttagcgaccctccctgcagggctgggcttttgcatggcaatggatgggacattaaagggacatgtaacaagcacaccggcctgctgttctgtccttccatccctcttttgggctcttctggagggaagtaacatttactgagcacctgttgtatgtcacatgccttatgaatagaatcttaactcctagagcaactctgtggggtggggaggagcagatccaagttttgcggggtctaaagctgtgtgtgttgagggggatactctgtttatgaaaaagaataaaaaacacaaccacgaaaaaaaaaaaaaaaaaaaaaaa

SEQ ID NO: 18 - protein, 461 aa, Homo sapiens
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVCLGEYDLRRWEKWELCLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP